(19) United States
(12) Patent Application Publication
(10) Pub. No.: US 2025/0177150 A1
(43) Pub. Date: Jun. 5, 2025

(54) ORTHOPEDIC IMPLANT ASSEMBLY FOR PROXIMAL FEMUR REPLACEMENT

(71) Applicants: Smith & Nephew, Inc., Memphis, TN (US); Smith & Nephew Orthopaedics AG, Zug (CH); Smith & Nephew Asia Pacific PTE. Limited, Singapore (SG)

(72) Inventors: Matthew J. Quick, Memphis, TN (US); Roger R. Dees, Jr., Drummonds, TN (US); Randy C. Winebarger, Southhaven, MS (US); Kaytlyn Thomas, Memphis, TN (US); Michael L. Cole, Collierville, TN (US)

(73) Assignees: Smith & Nephew, Inc., Memphis, TN (US); Smith & Nephew Orthopaedics AG, Zug (CH); Smith & Nephew Asia Pacific PTE. Limited, Singapore (SG)

(21) Appl. No.: 18/843,794

(22) PCT Filed: Mar. 14, 2023
(86) PCT No.: PCT/US2023/015187

§ 371 Date: Sep. 4, 2024

Related U.S. Application Data
(60) Provisional application No. 63/324,117, filed on Mar. 27, 2022.
(60) Provisional application No. 63/324,118, filed on Mar. 27, 2022.

(51) Int. Cl. A61F 2/36; A61B 17/00; A61B 17/80; A61F 2/30

(52) U.S. Cl. CPC A61F 2/3607; A61B 17/8066; A61B 17/809; A61F 2/30749; A61B 2017/00477; A61F 2002/30433; A61F 2002/3613

(57) ABSTRACT

A proximal femur replacement implant assembly (500, 1000) arranged and configured to replace a head portion of a patient's proximal femur is disclosed. In some examples, the proximal femur replacement implant assembly includes a proximal femur replacement implant (505, 1005) arranged and configured to be coupled to, or inserted into an intramedullary canal of, a remaining portion of a patient's femur. The proximal femur replacement implant being arranged and configured to engage a bone plate (510, 1010, 1050) to, for example, minimize micromotion of the patient's greater trochanter to promote porous in-growth and to constrain movement of the greater trochanter.

### CROSS-REFERENCE TO RELATED APPLICATIONS

This is a non-provisional of, and claims the benefit of the filing date of, U.S. provisional patent application No. 63/324,117, filed Mar. 27, 2022, entitled “Orthopedic Implant Assembly for Total Proximal Femur Replacement” and is a non-provisional of, and claims the benefit of the filing date of, U.S. provisional patent application No. 63/324,118, filed Mar. 27, 2022, entitled “Bone Plate,” the entirety of each application is incorporated by reference herein.

### FIELD OF THE DISCLOSURE

The present disclosure relates generally to orthopedic devices and more particularly to orthopedic implant assemblies, bone plates, and other components and instruments, and corresponding methods and systems, for proximal femur replacement.

### BACKGROUND OF THE DISCLOSURE

Orthopedic implants are well known and commonplace in today's society. Orthopedic implants may be used, for example, to stabilize an injury, to support a bone fracture, to fuse a joint, and/or to correct a deformity. In some instances, orthopedic implants may even be used to replace one or more bones, bone pieces, bone portions, or bone segments (terms used interchangeably without the intent to limit). Orthopedic implants may be attached permanently or temporarily, and may be attached to the bone at various locations, including implanted within a canal or other cavity of the bone, implanted beneath soft tissue and attached to an exterior surface of the bone, or disposed externally and attached by fasteners such as screws, pins, and/or wires. Some orthopedic implants allow the position and/or orientation of two or more bone pieces, or two or more bones, to be adjusted relative to one another.

One such orthopedic implant is a femoral hip stem implant used to repair a hip joint. The hip joint is a frequent place for joint damage and/or injury. Femoral hip stem implants can be implanted or otherwise associated with the bony anatomy for treating traumatic injuries, reconstructing joint function, or for other purposes. Femoral hip stem implants may include an elongated insertion region, such as a stem region, which can be at least partially inserted into an intramedullary canal of a patient's proximal femur.

In some instances, the entire head portion of the patient's femur may be replaced by a proximal femur replacement or greater trochanter implant (terms used interchangeably herein). In such procedures, the proximal femur may be resected and the intramedullary canal of the remaining femur may be prepared. The proper implant assembly may be determined and inserted.

In some examples, the patient's greater trochanter may then be coupled to the proximal femur replacement implant via sutures or ligatures. However, the micromotion experienced from coupling the greater trochanter to the proximal femur replacement implant using sutures alone is greater than what is typically desired to promote porous ingrowth. Additionally, sutures are less effective in constraining movement of the greater trochanter in the anterior/posterior direction.

Alternatively, in other procedures, the addition of a bone plate may be used to secure the patient's bone and/or soft tissue. That is, with reference to FIG. 1, a proximal femur replacement assembly 10 may be utilized. The proximal femur replacement assembly 10 includes a proximal femur replacement implant 20, a bone plate 30, and a bolt 40 arranged and configured to couple the bone plate 30 to the proximal femur replacement implant 20.

Current proximal femur replacement assemblies suffer from several disadvantages. First, because the bone plate 30 is coupled to the proximal femur replacement implant 20 via a bolt 40, the height and/or position of the bone plate 30 is fixed relative to the proximal femur replacement implant 20. In addition, because of the bolt connection, the bone plate 30 may be positioned too close or too far from the proximal femur replacement implant 20. If the bone plate 30 is positioned too close to the proximal femur replacement implant 20, the patient's bone may be compressed between the bone plate 30 and the proximal femur replacement implant 20. If the bone plate 30 is positioned too far away, the assembly may suffer from decreased fixation.

Another well-known orthopedic implant is a bone plate. Bone plates are often used to fix one or more patient's bone(s). In orthopedic surgeries, it is not uncommon for a patient to receive a bone plate to repair one or more fractures in a patient's bone including, for example, in proximal femur replacement procedures as described above in connection with FIG. 1.

As mentioned in connection with FIG. 1, on occasion a bone plate may be used in combination with other orthopedic implants. For example, during a hip replacement procedure or a proximal femur replacement procedure, a bone plate may be needed adjacent to a femoral hip stem implant used to repair the patient's hip joint.

Another common procedure where a bone plate may be needed adjacent to, or in close proximity to, an orthopedic implant is a knee replacement or knee arthroplasty.

When performing a proximal femoral replacement (e.g., hip replacement procedure) or a proximal tibial replacement (e.g., a knee replacement) one of the most difficult tasks for surgeons is to reattach whatever remaining bone portions and/or soft tissue that are available. The variability in what is left is part of what makes this task so difficult.

Currently, a variety of sutures and/or fixed design (e.g., non-customizable) bone plates may be used to reattach any remaining bone portions or soft tissue. However, utilization of sutures and/or non-customizable bone plates suffer from several disadvantages. For example, the use of sutures typically provides micromotion that is greater than desired to promote porous ingrowth (e.g., sutures are less effective in constraining movement). Utilization of non-customizable bone plates tend to provide a “one size fits all” method to reattaching any remaining bone portions or soft tissues (e.g., utilization of non-customizable bone plates minimize surgeon's flexibility to tailor the implant assembly to the individual patient needs).

Thus, it would be beneficial to provide a proximal femur replacement assembly that provides increased flexibility in the positioning of the bone plate relative to the proximal femur replacement implant so that surgeons can better tailor the implant assembly to the individual patient needs. In addition, it would be beneficial to provide a bone plate, and more specifically, a customizable bone plate that provides increased flexibility and/or tailoring in the positioning of the bone plate relative to the patient's remaining bone and/or other orthopedic implants such as, for example, proximal femur replacement, orthopedic hip implants, orthopedic knee implants, etc.

It is with this in mind that the present disclosure is provided.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In some examples, the present disclosure is directed to a proximal femur replacement implant assembly arranged and configured to replace a head portion of a patient's proximal femur. In some examples, the proximal femur replacement implant assembly includes a proximal femur replacement implant arranged and configured to be coupled to, or inserted into an intramedullary canal of, a remaining portion of a patient's femur, a primary bone plate arranged and configured to be coupled to the patient's bone and/or soft tissue, and a secondary bone plate arranged and configured to couple the primary bone plate to the proximal femur replacement implant.

In any preceding or subsequent example, the proximal femur replacement implant includes a proximal end, a distal end, a first side surface, a second side surface, a neck extending from the proximal end, and an intramedullary stem extending from the distal end. The neck being arranged and configured to receive a femoral head configured to interact with an acetabular system and/or hip socket. The intramedullary stem being arranged and configured to be inserted into an intramedullary canal of a patient's bone (e.g., femur). The proximal femur replacement implant further including an attachment mechanism arranged and configured to couple the secondary bone plate to the proximal femur replacement implant.

In any preceding or subsequent example, the attachment mechanism includes one or more screw fixation holes formed in the first and second side surfaces of the proximal femur replacement implant, each of the one or more screw fixation holes arranged and configured to receive a fastener or screw for coupling the secondary bone plate to the first and second side surfaces of the proximal femur replacement implant.

In any preceding or subsequent example, the first and second side surfaces of the proximal femur replacement implant each include a recessed surface, the one or more screw fixation holes being formed in the recessed surface. The recessed surfaces being arranged and configured to be recessed a sufficient distance to reduce the extent or distance that the secondary bone plate and/or fasteners extend from the outer surface of the proximal femur replacement implant to minimize associated patient trauma.

In any preceding or subsequent example, the primary bone plate includes a hook-shaped plate including one or more hooks formed on a proximal end thereof configured to engage a patient's proximal femur.

In any preceding or subsequent example, the secondary bone plate includes a first end, a second end, and a central portion extending between the first and second ends. The secondary bone plate including a plurality of holes or openings formed therein arranged and configured to enable a fastener to pass therethrough. Thus arranged, the first end of the secondary bone plate may be coupled to the first side surface of the proximal femur replacement implant via one or more fasteners passing through one of the holes or openings formed in the first end of the secondary bone plate and into one of the screw fixation holes formed in the first side surface of the proximal femur replacement implant. Thereafter, the secondary bone plate may be bent or angled so that the central portion of the secondary bone plate extends across a surface (e.g., top surface) of the primary bone plate. Next, the secondary bone plate may be bent again so that the second end of the secondary bone plate may be coupled to the second side surface of the proximal femur replacement implant via one or more fasteners passing through one of the holes or openings formed in the second end of the secondary bone plate and into one of the screw fixation holes formed in the second side surface of the proximal femur replacement implant.

In any preceding or subsequent example, the secondary bone plate may be pressed-fitted to the primary bone plate.

In any preceding or subsequent example, the proximal femur replacement implant assembly may further include a bolt arranged and configured to couple the secondary bone plate to the proximal femur replacement implant.

In addition, in some examples, the present disclosure is directed to a bone plate, and more particularly, a customizable bone plate. That is, in accordance with one or more features of the present disclosure, the bone plates are arranged and configured to be flexible, and thus customizable, in order to allow surgeons to adjust the position, orientation, configuration, etc. of the bone plate to fit the individual patient needs.

In any preceding or subsequent example, the customizable bone plate may be particularly well-suited for use in connection with proximal femur and/or proximal tibia replacements or surgical procedures.

In any preceding or subsequent example, the customizable bone plate includes a center hub or connection point. In addition, the customizable bone plate includes a plurality of arms, tines, spokes, or segments extending from the center hub or connection point. Thus arranged, the customizable bone plate may be referred to as a hub-and-spoke design or a spider plate.

In any preceding or subsequent example, the center hub or connection point can be directly connected to, or placed in contact with, the patient's remaining bone and/or to the previously implanted proximal tibia or femoral implant. The plurality of arms may be rendered flexible or bendable so that they can be repositioned to couple or encompass the patient's remaining bone fragments and/or other orthopedic implants to provide increased versatility to secure the patient's remaining bone fragments.

Additionally, in some examples, the plurality of arms may be arranged and configured to be removed (e.g., cut) as well as bent to enable increased flexibility in shaping the bone plate as needed.

In some examples of use, the customizable bone plate may be used in a proximal femur replacement implant assembly arranged and configured to replace a head portion of a patient's proximal femur. In some examples, the proximal femur replacement implant assembly includes a proximal femur replacement implant arranged and configured to be coupled to, or inserted into an intramedullary canal of, a remaining portion of a patient's femur and a customizable bone plate arranged and configured to be coupled to the patient's bone and/or soft tissue, and to the proximal femur replacement implant.

In any preceding or subsequent example, the proximal femur replacement implant includes a proximal end, a distal end, a first side surface, a second side surface, a neck extending from the proximal end, and an intramedullary stem extending from the distal end. The neck being arranged and configured to receive a femoral head configured to interact with an acetabular system and/or hip socket. The intramedullary stem being arranged and configured to be inserted into an intramedullary canal of a patient's bone (e.g., femur). The proximal femur replacement implant further including an attachment mechanism arranged and configured to couple the customizable bone plate to the proximal femur replacement implant.

In any preceding or subsequent example, the attachment mechanism includes one or more screw fixation holes formed in the first and second side surfaces of the proximal femur replacement implant, each of the one or more screw fixation holes arranged and configured to receive a fastener or screw for coupling the customizable bone plate to the first and second side surfaces of the proximal femur replacement implant.

In any preceding or subsequent example, the first and second side surfaces of the proximal femur replacement implant each include a recessed surface, the one or more screw fixation holes being formed in the recessed surface. The recessed surfaces being arranged and configured to be recessed a sufficient distance to reduce the extent or distance that the customizable bone plate and/or fasteners extend from the outer surface of the proximal femur replacement implant to minimize associated patient trauma.

In another example of use, the customizable bone plate may be used in a proximal tibia replacement implant assembly arranged and configured to replace a head portion of a patient's proximal tibia. In some examples, the proximal tibia replacement implant assembly includes a proximal tibia replacement implant arranged and configured to be coupled to a remaining portion of a patient's tibia and a customizable bone plate arranged and configured to be coupled to the patient's bone and/or soft tissue, and to the proximal tibia replacement implant.

In any preceding or subsequent example, the proximal tibia replacement implant includes a proximal end, a distal end, a first side surface and a second side surface. The proximal end of the proximal tibia replacement implant defining a top surface arranged and configured to be operatively associated with a knee prosthesis. The proximal tibia replacement implant further including an intramedullary stem extending from the distal end. The intramedullary stem being arranged and configured to be inserted into an intramedullary canal of a patient's bone (e.g., tibia). In addition, the proximal tibia replacement implant further includes an attachment mechanism arranged and configured to couple the customizable bone plate to the proximal tibia replacement implant.

In any preceding or subsequent example, the attachment mechanism includes one or more screw fixation holes formed in the first and second side surfaces of the proximal tibia replacement implant, each of the one or more screw fixation holes arranged and configured to receive a fastener or screw for coupling the customizable bone plate to the first and second side surfaces of the proximal tibia replacement implant.

In an alternate example, a proximal femur replacement implant assembly arranged and configured to replace a head portion of a patient's proximal femur is disclosed. The assembly comprising a proximal femur replacement implant arranged and configured to be coupled to, or inserted into an intramedullary canal of, a remaining portion of a patient's femur, the proximal femur replacement implant including a proximal end, a distal end, a first side surface, a second side surface, a medial side surface, a lateral side surface, and a bridge member spaced from the lateral side surface.

In any preceding or subsequent example, the bridge member includes an implant facing surface, an outer surface opposite the implant facing surface, and a plurality of openings formed therein for receiving a plurality of fasteners.

In any preceding or subsequent example, the plurality of openings include one or more locking screw openings and one or more variable angled openings.

In any preceding or subsequent example, the bridge member is integrally and monolithically formed with the proximal femur replacement implant.

In any preceding or subsequent example, the bridge member includes a porous surface formed on a portion thereof.

In any preceding or subsequent example, the assembly further includes a bone plate and the plurality of fasteners for coupling the bone plate to the bridge member.

In any preceding or subsequent example, the assembly further includes the plurality of fasteners, wherein at least one of the plurality of fasteners comprises a cable saddle screw including a threaded shaft and a head portion, the head portion including a plurality of transverse openings arranged and configured to enable a cable, a suture, or a ligament to pass therethrough.

In any preceding or subsequent example, at least one of the plurality of openings formed in the bridge member is arranged and configured to engage the threaded shaft of the cable saddle screw.

In any preceding or subsequent example, the bridge member includes one or more spikes or projections extending laterally therefrom to facilitate initial fixation.

Examples of the present disclosure provide numerous advantages. For example, by utilizing a proximal femur replacement implant assembly as described herein, increased flexibility in positioning a bone plate relative to the proximal femur replacement implant is provided thus enabling a surgeon to better tailor the assembly to individual patient's needs. In addition, when utilizing a proximal femur replacement implant assembly as described herein during a limb salvage surgery, the minimal amount of patient's bone remaining can be preserved. For example, the remaining bone can be preserved through contact with a porous surface on an implant or component thereof. In addition, and/or alternatively, when reattaching the patient's greater trochanter to the proximal femur replacement implant assembly, motion can be better constrained to, for example, limit the anterior/posterior and medial/lateral translation in a way that retains the proximal/distal and medial/lateral constraints, while also minimizing micromotion to better accommodate porous ingrowth between the preserved bone and the surgical implants.

In addition, and/or alternatively, by providing surgeons with one or more customizable bone plates in, for example, a kit, the bone plates provide surgeons with increased flexibility to adjust the configuration of the bone plate for each individual patient's needs

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1 illustrates a conventional proximal femur replacement implant assembly;

FIG. 2 illustrates a perspective view of an example of a proximal femur replacement implant assembly in accordance with one or more features of the present disclosure;

FIG. 3A illustrates an alternate perspective view of the proximal femur replacement implant assembly shown in FIG. 2;

FIG. 3B illustrates a side (e.g., medial/lateral) view of the proximal femur replacement implant assembly shown in FIG. 2;

FIGS. 4A and 4B illustrate various schematic views of the proximal femur replacement implant assembly shown in FIG. 2;

FIG. 5 illustrates a top view of an example of a customizable bone plate in accordance with one or more features of the present disclosure;

FIG. 6 illustrates a top view of an alternate example of a customizable bone plate in accordance with one or more features of the present disclosure;

FIG. 7 illustrates a top view of an alternate example of a customizable bone plate in accordance with one or more features of the present disclosure;

FIG. 8 illustrates a top view of an alternate example of a customizable bone plate in accordance with one or more features of the present disclosure;

FIG. 9 illustrates a top view of an alternate example of a customizable bone plate in accordance with one or more features of the present disclosure;

FIG. 10 illustrates a top view of an alternate example of a customizable bone plate in accordance with one or more features of the present disclosure;

FIG. 11 illustrates a top view of an alternate example of a customizable bone plate in accordance with one or more features of the present disclosure;

FIG. 12 illustrates a top view of an alternate example of a customizable bone plate in accordance with one or more features of the present disclosure;

FIG. 13 illustrates a top view of an alternate example of a customizable bone plate in accordance with one or more features of the present disclosure;

FIG. 14A illustrates a perspective view of an example of a proximal femur replacement implant assembly including a proximal femur replacement implant and a customizable bone plate in accordance with one or more features of the present disclosure;

FIG. 14B illustrates an alternate perspective view of the proximal femur replacement implant assembly shown in FIG. 14A;

FIG. 15A illustrates a perspective view of an example of a proximal tibia replacement implant assembly including a proximal tibia replacement implant and a customizable bone plate in accordance with one or more features of the present disclosure;

FIG. 15B illustrates an alternate perspective view of the proximal tibia replacement implant assembly shown in FIG. 15A;

FIG. 16 illustrates a perspective view of an alternate example of a proximal tibia replacement implant assembly including a proximal tibia replacement implant and a customizable bone plate in accordance with one or more features of the present disclosure;

FIGS. 17A-17C illustrate various views of an alternate example of a proximal femur replacement implant assembly in accordance with one or more features of the present disclosure;

FIGS. 18A and 18B illustrate various views of an alternate example of a proximal femur replacement implant assembly in accordance with one or more features of the present disclosure;

FIGS. 19A and 19B illustrate various views of an alternate example of a proximal femur replacement implant assembly in accordance with one or more features of the present disclosure;

FIG. 19C illustrates a side view of an example method of use utilizing a proximal femur replacement implant assembly in accordance with one or more features of the present disclosure;

FIG. 20 illustrates a perspective view of an alternate example of a proximal femur replacement implant assembly in accordance with one or more features of the present disclosure;

FIGS. 21A-21C illustrate various views of an example of an alignment instrument that may be used in connection with a proximal femur replacement implant assembly in accordance with one or more features of the present disclosure;

FIGS. 22A-22D illustrate various views of a method of implanting a proximal femur replacement implant assembly in accordance with one or more features of the present disclosure; and

FIGS. 23A and 23B illustrate various views of an alternate example of a proximal femur replacement implant assembly in accordance with one or more features of the present disclosure.

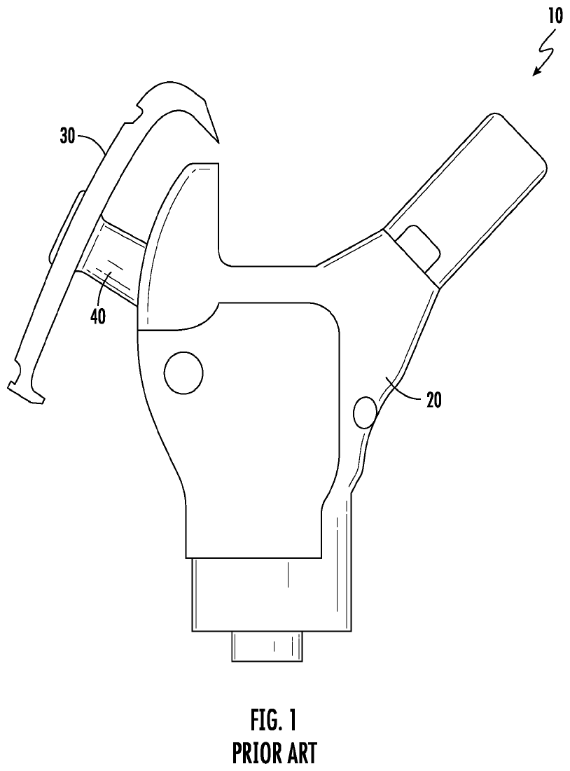

The drawings are not necessarily to scale. The drawings are merely representations, not intended to portray specific parameters of the disclosure. The drawings are intended to depict various examples of the disclosure, and therefore are not to be considered as limiting in scope. In the drawings, like numbering represents like elements.

### DETAILED DESCRIPTION

Various features of a proximal femur replacement or greater trochanter implant assembly (terms used interchangeably herein) will now be described more fully herein with reference to the accompanying drawings, in which one or more features of the proximal femur replacement implant assembly will be shown and described. It should be appreciated that the various features may be used independently of, or in combination, with each other. It will be appreciated that a proximal femur replacement implant assembly as disclosed herein may be embodied in many different forms and may selectively include one or more features described herein. As such, the proximal femur replacement implant assembly should not be construed as being limited to the specific examples set forth herein. Rather, these examples are provided so that this disclosure will convey certain features of the proximal femur replacement implant assembly to those skilled in the art.

As will be described in greater detail herein, in accordance with one or more features of the present disclosure, the proximal femur replacement implant assembly includes a proximal femur replacement implant arranged and configured to replace, or at least partially replace, the head portion or proximal end of a patient's femur. The proximal femur replacement implant is arranged and configured to be implanted within an intramedullary canal of the patient's femur. In addition, the proximal femur replacement implant assembly includes a primary bone plate arranged and configured to be coupled to the patient's bone and/or soft tissue, and a secondary bone plate arranged and configured to couple the primary bone plate to the proximal femur replacement implant. In use, the secondary bone plate enables increased flexibility in positioning of the primary bone plate relative to the proximal femur replacement implant thus enabling a surgeon to better tailor the assembly to individual patient's needs.

That is, as will be described in greater detail herein, in use, in accordance with one or more features of the present disclosure, a secondary bone plate is used to couple the primary bone plate to the proximal femur replacement implant. Utilization of a secondary bone plate, which may be arranged and configured to extend across a surface (e.g., top surface) of the primary bone plate, enables increased flexibility in positioning of the primary bone plate relative to the proximal femur replacement implant. In addition, since the secondary bone plate may be arranged and configured to couple to the proximal femur replacement implant on either side thereof, the assembly can be used on either the left or right proximal femur thus eliminating the need to manufacture and provide handed proximal femur replacement implants.

Thus arranged, the greater trochanter may be reattached to the proximal femur replacement implant with sufficient constraint to allow for porous ingrowth and overall system longevity.

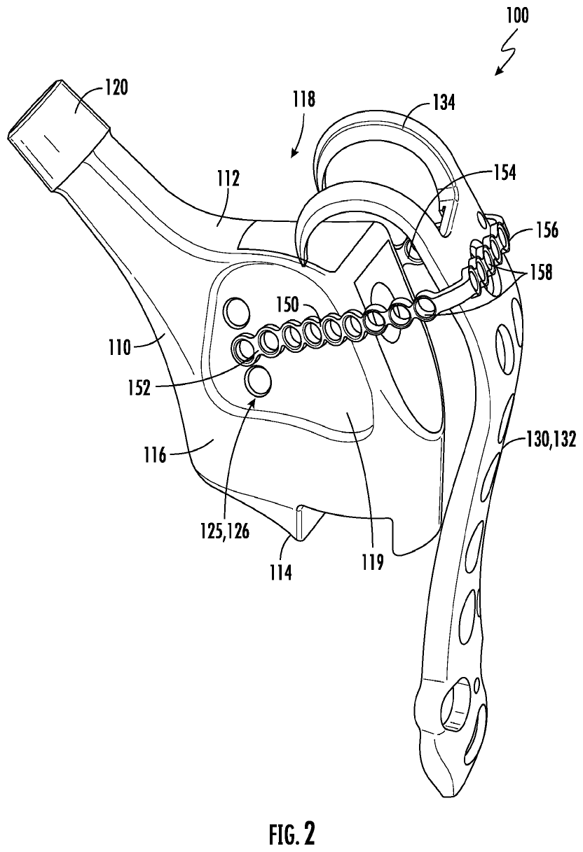

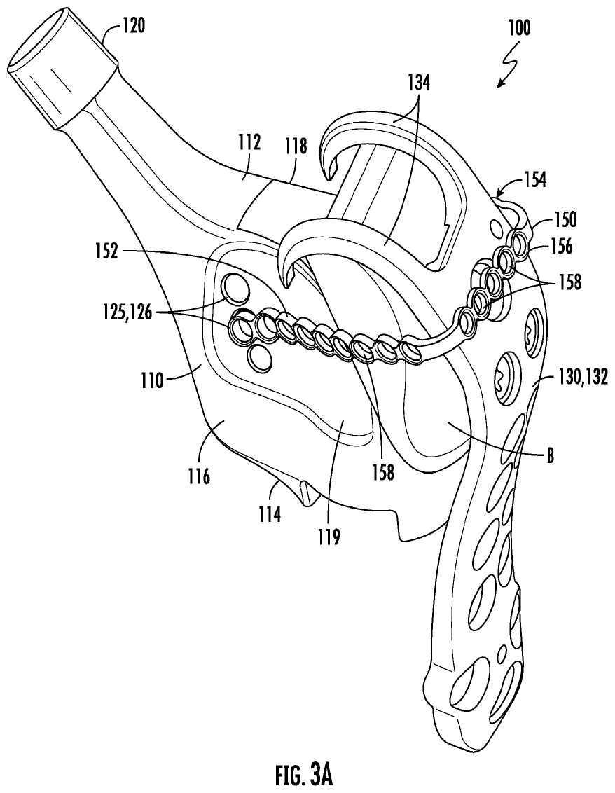

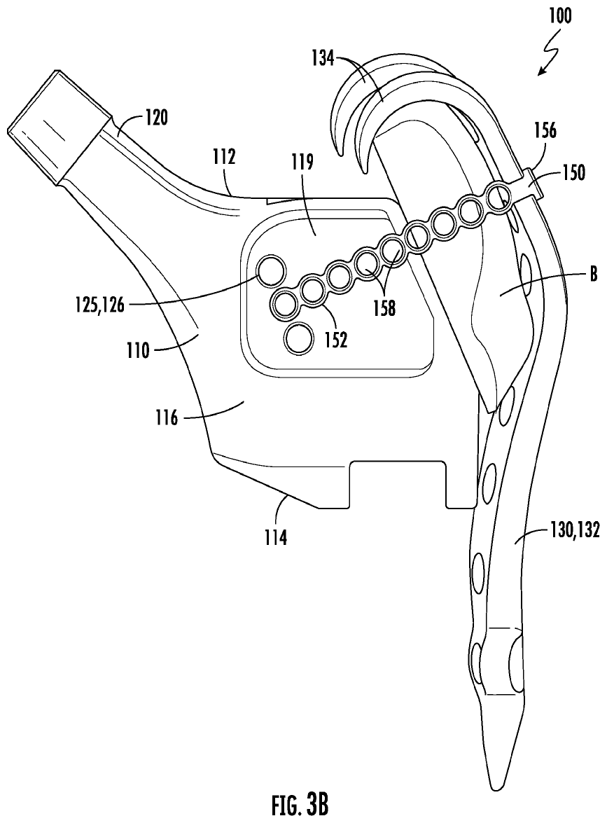

Referring to FIGS. 2-4B, an example of a proximal femur replacement implant assembly 100 is disclosed. As disclosed, the proximal femur replacement implant assembly 100 includes a proximal femur replacement implant 110, a primary bone plate 130, and a secondary bone plate 150 arranged and configured to couple the primary bone plate 130 to the proximal femur replacement implant 110.

As illustrated, the proximal femur replacement implant 110 includes a proximal end 112, a distal end 114, a first side surface 116, and a second side surface 118. In use, the proximal femur replacement implant 110 includes a neck 120 extending from the proximal end 112. In use, as will be appreciated by one of ordinary skill in the art, the neck 120 is arranged and configured to receive a femoral head configured to interact with an acetabular system and/or hip socket. In addition, and as will be appreciated by one of ordinary skill in the art, the proximal femur replacement implant 110 includes an intramedullary stem arranged and configured to be inserted into an intramedullary canal of a patient's bone (e.g., femur). As will be appreciated by one of ordinary skill in the art, in use, the patient's femur is initially prepared for implantation of the proximal femur replacement implant 110 by, for example, resecting the proximal femur and reaming the intramedullary canal in the patient's femur. The stem of the proximal femur replacement implant 110 is then inserted into the intramedullary canal of the resected femur.

In accordance with one or more features of the present disclosure, the proximal femur replacement implant 110 includes an attachment mechanism 125 arranged and configured to couple the secondary bone plate 150 to the proximal femur replacement implant 110. In use, the attachment mechanism 125 may be any suitable mechanism now known or hereafter developed. For example, as illustrated, the attachment mechanism 125 may be provided in the form of one or more screw fixation holes 126 formed in the first and second side surfaces 116, 118. In use, and as will be described in greater detail below, the one or more screw fixation holes 126 are arranged and configured to receive one or more fasteners, respectively, for coupling the secondary bone plate 150 to the first and second side surfaces 116, 118 of the proximal femur replacement implant 110.

As illustrated, in some examples, the proximal femur replacement implant 110 may include a plurality of screw fixation holes 126 such as, for example, three screw fixation holes. However, it should be appreciated that any number and arrangement of screw fixation holes may be provided including, more or less screw fixation holes arranged in any arrangement. In addition, while the screw fixation holes are shown as being internally threaded, it should be appreciated that the screw fixation holes may be provided in any now known or hereafter developed configuration including, for example, including a plurality of fins arranged and configured to enable the screw fastener to be angulated relative to the proximal femur replacement implant 110. That is, one or more of the screw fixation holes could include fins and/or slots to allow for multiple positions at various angles.

As illustrated, in some examples, the first and second side surfaces 116, 118 of the proximal femur replacement implant 110 may each include a recessed surface 119 with the plurality of screw fixation holes 126 positioned within the recessed surfaces 119. In use, the recessed surface 119 may be the same on either side 116, 118 of the implant 110 to prevent the need for handed implants. Thus arranged, the recessed surface 119 may be arranged and configured to be recessed a sufficient distance to prevent the secondary bone plate 150 and/or fasteners from extending a greater distance beyond an outer surface of the non-recessed surface of the implant 110 thereby minimizing associated patient trauma (e.g., the recessed surfaces 119 reduce the extent or distance that the secondary bone plate 150 and/or fasteners extend from the outer surface of the proximal femur replacement implant 110 to minimize associated patient trauma).

It should be appreciated that the present disclosure is directed to a proximal femur replacement implant assembly 100 arranged and configured to provide increased flexibility in positioning the primary bone plate 130 relative to the proximal femur replacement implant 110. In use, while a particular proximal femur replacement implant 110 is shown and described, it should be understood that any proximal femur replacement implant 110 now known or hereafter described can be used including, for example, it is envisioned that a conventional femoral stem may be used in place of the proximal femur replacement implant. As such, the present disclosure should not be limited to any particular proximal femur replacement implant 110. Thus, for the sake of brevity, additional detailed discussion on the proximal femur replacement implant 110 is omitted herefrom. Moreover, while examples of the present disclosure are described and illustrated in connection with a proximal femur or a hip implant, examples of the present disclosure may be equally applicable to other orthopedic implants including, for example, shoulder implants, ankle implants, etc.

As previously mentioned, the proximal femur replacement implant assembly 100 also includes a primary bone plate 130. In use, the primary bone plate 130 may be any suitable bone plate now known or hereafter developed. For example, as illustrated, the primary bone plate 130 may be provided in the formed of a hook-shaped plate 132. As will be appreciated by one of ordinary skill in the art, hook-shaped plates include one or more hooks 134 formed on a proximal end thereof configured to engage a patient's proximal femur B. In use, the hook-shaped plate 132 can be used in conjunction with the proximal femur replacement implant 110 to better secure the greater trochanter to the implant's porous surfaces. However, it should be appreciated that while a particular primary bone plate is shown and described, it should be understood that the primary bone plate may have any configuration now known or hereafter developed including, for example, proximal femur plates, a plate including multiple reconfigurable arms, etc. As such, the present disclosure should not be limited to any particular configuration or style of bone plate. Thus, for the sake of brevity, additional detailed discussion on the primary bone plate is omitted herefrom.

With reference to FIGS. 2-4B, in accordance with one or more features of the present disclosure, the proximal femur replacement implant assembly 100 includes a secondary bone plate 150 arranged and configured to couple the primary bone plate 130 to the proximal femur replacement implant 110. As illustrated, in some examples, the secondary bone plate 150 may be provided in the form of a longitudinal plate including a plurality of holes or openings 158 formed therein arranged and configured to enable a fastener to pass therethrough. That is, as illustrated, the secondary bone plate 150 may include a first end 152, a second end 154, and a central portion 156 extending between the first and second ends 152, 154. As illustrated, in some examples, the plurality of holes or openings 158 may be provided along an entire, or substantially entire, longitudinal length of the secondary bone plate 150. In addition, in some examples, the secondary bone plate 150 is arranged and configured to bend so that in use, as illustrated, the first end 152 of the secondary bone plate 150 can be coupled to one of the side surfaces such as, for example, the first side surface 116, of the proximal femur replacement implant 110, the central portion 156 can be positioned adjacent to, extend across, wrap around, etc., the primary bone plate 130, and the second end 154 of the secondary bone plate 150 can then be coupled to the other one of the side surfaces such as, for example, the second side surface 118, of the proximal femur replacement implant 110.

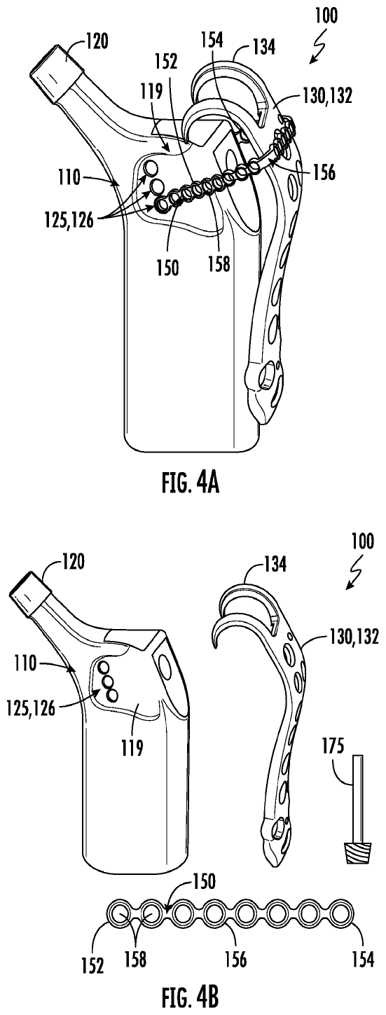

Thus arranged, as illustrated, in accordance with one or more features of the present disclosure, an additional (e.g., secondary) bone plate 150 can be coupled to the proximal femur replacement implant 110 via one or more fasteners utilizing one or more of the plurality of screw fixation holes 126. For example, a first end 152 of the secondary bone plate 150 can be coupled to one of the side surfaces of the proximal femur replacement implant 110 such as, for example, the first side surface 116 of the proximal femur replacement implant 110. The secondary bone plate 150 can then be bent so that the central portion 156 of the secondary bone plate 150 can extend across a surface of the primary bone plate 130 such as, for example, across a top surface of the primary bone plate 130, and then bent again so that the second end 154 of the secondary bone plate 150 can be coupled to the opposing side surface of the proximal femur replacement implant 110 such as, for example, the second side surface 118, via one or more fasteners utilizing one or more of the plurality of screw fixation holes 126. For example, in use, an instrument 175 (FIG. 4B) can be attached to the desired screw fixation hole 126 formed in the proximal femur replacement implant 110 (e.g., an instrument 175 having a threaded end portion can be threadably coupled to one of the screw fixation holes 126). Thereafter, the secondary bone plate 150 can be slid over the instrument 175 that is coupled to the implant 110 and bent around the primary bone plate 130 (e.g., hook plate). A second threaded instrument 175 can then be used on the opposing side of the proximal femur replacement implant 110 to ensure the location of the secondary bone plate 150 is aligned between the proximal femur replacement implant 110 and the primary bone plate 130 (e.g., hook plate). Once everything is properly positioned, the surgeon can remove the instruments 175 one at a time and insert a fastener to secure the secondary bone plate 150 to the proximal femur replacement implant 110.

In some examples, the secondary bone plate 150 may be pressed-fitted to and/or against the primary bone plate 130. That is, no physical screw connection between the secondary and primary bone plate is required. Alternatively, however, the secondary bone plate 150 may be coupled to the primary bone plate 130 via one or more fasteners.

In addition, in some examples, the secondary bone plate 150 can be used in combination with a bolt arranged and configured to directly engage the primary bone plate 130 to the proximal femur replacement implant 110.

In accordance with features of the present disclosure, by utilizing a secondary bone plate 150 to couple the primary bone plate 130 to the proximal femur replacement implant 110, the proximal femur replacement implant assembly 100 provides surgeons with the ability to tailor the assembly 100 as needed to, for example, fix any remaining bone and/or soft tissue (e.g., provides increased flexibility in determining proper fixation positioning). For example, variation in height or positioning of the primary bone plate 130 relative to the proximal femur replacement implant 110 can be accommodated thus providing surgeons with increased flexibility to reposition the primary bone plate 130 with respect to the proximal femur replacement implant 110. In addition, by coupling the primary bone plate 130 to the proximal femur replacement implant 110 via the secondary bone plate 150, the assembly 100 prevents, or at least minimizes, superior/inferior and anterior/posterior movements of the bone B. As illustrated herein, bone B may be a piece or portion of a patient's bone such as an intact piece of bone or a chipped bone fragment. Alternatively, the bone B may be a piece of allograft, bone slurry, bone putty, etc. That is, utilization of the secondary bone plate 150 enables a surgeon to reposition the patient's remaining bone B in the superior-inferior direction and the medial-lateral direction, then once the bone B is properly positioned, the surgeon can secure the position of the various components, and hence the patient's bone B and/or soft tissue. Thus arranged, the assembly 100 can limit anterior/posterior translation while retaining proximal/distal and medial/lateral constraints, while also minimizing micromotion to better accommodate porous ingrowth between the preserved bone B and surgical implants.

In some examples, it is envisioned that the proximal femur replacement implant assembly 100 can also provide a retention mechanism to enable allograft to be used in-between the primary bone plate 130 and the proximal femur replacement implant 110. Thus arranged, improved bone-in-growth can be achieved.

Additionally, while only a single secondary bone plate 150 has been shown and described, in alternate examples, utilization of multiple secondary bone plates 150 to couple the primary bone plate 130 to the proximal femur replacement implant 110 is envisioned.

Alternatively, in some examples, it is envisioned that the primary bone plate 130 can be eliminated entirely and the secondary bone plate 150 can be used to bend around the patient's bone (e.g., greater trochanter) while connected to the proximal femur replacement implant 110. Moreover, in some examples, as will be described and illustrated in alternate examples, the primary and the secondary bone plates may be combined, or monolithically formed. In other examples, the primary and secondary bone plates may be separately formed and coupled to each other (and/or to the implant) prior to or during surgery.

### Bone Plates

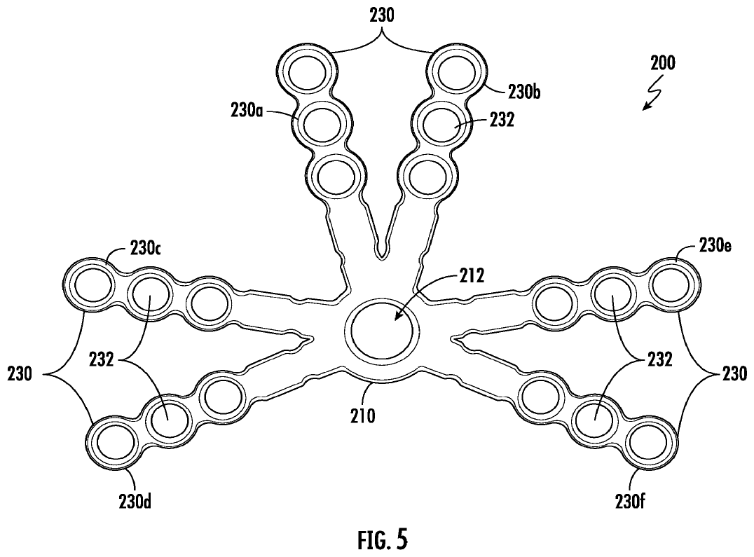

With reference to FIGS. 5-13, various features of an improved bone plate will now be described more fully herein, in which one or more features of the bone plates will be shown and described. In use, the bone plates can be used in a proximal femur replacement implant assembly. Alternatively, however, the bone plates may be used in other surgical procedures to fix bones in other patient's areas. Moreover, one or more corresponding orthopedic assemblies and/or methods of use will also be described more fully herein with reference to the accompanying drawings including, for example, a proximal femur replacement implant assembly and a proximal tibia replacement implant assembly, although it should be appreciated that the bone plates may be used in any orthopedic surgical application. It should be appreciated that the various features may be used independently of, or in combination, with each other. It will be appreciated that a bone plate as disclosed herein may be embodied in many different forms and may selectively include one or more features described herein. As such, the bone plate should not be construed as being limited to the specific examples set forth herein. Rather, these examples are provided so that this disclosure will convey certain features of the bone plate to those skilled in the art.

With reference to FIGS. 5-13, in accordance with one or more features of the present disclosure, various bone plates arranged and configured with increased flexibility and/or customizability to enable surgeons to adjust the configuration of the bone plate to each individual patient's needs will be described. In some examples, the bone plates include a center connection point or a center hub (terms used interchangeably herein) and a plurality of arms, tines, spokes, or segments (terms used interchangeably herein) extending from the center hub. Thus arranged, the customizable bone plate may be referred to as a hub-and-spoke design or a spider plate. In some examples, the center hub can be directly connected to, or placed in contact with, the patient's remaining bone and/or to a previously implanted orthopedic implant. The plurality of arms may be bent, for example, relative to the center hub, so that one or more of the plurality of arms may be repositioned and/or bent as needed to position the arms to encompass or engage the patient's remaining bone fragments and/or a previously implanted orthopedic implant. Thus arranged, the bone plates provide increased versatility to reposition portions of the bone plates as needed to secure the patient's remaining bone fragments.

Referring to FIG. 5, an example of a bone plate 200 in accordance with one or more features of the present disclosure is disclosed. As illustrated, the bone plate 200 includes a center hub 210 and a plurality of arms 230 extending from the center hub 210. As illustrated, in some examples, the center hub 210 may be provided with a circular shape, however this is but one configuration and the center hub 210 may have alternate shapes such as, for example, oval, square, or the like.

In addition, as illustrated, in some examples, the center hub 210 may include a hole or opening 212 formed therein. In use, the hole or opening 212 is arranged and configured to receive a screw or fastener to secure the center hub 210 to, for example, a patient's bone and/or a previously implanted orthopedic implant. It should be appreciated that while the center hub 210 is shown with one hole or opening 212, the center hub 210 may include any number of holes or openings provided in any arrangement or configuration including, for example, two, three, or more holes or openings. Alternatively, the center hub 210 may be completely devoid of any hole or opening. Moreover, while a particular example of a hole or opening is shown, it should be appreciated that the hole or opening may have any now known or hereafter developed configuration. For example, the hole or opening may include threads for threadably engaging a head portion of the screw or fastener (e.g., hole or opening may be configured as a locking screw opening). Alternatively, the hole or opening may be arranged and configured to enable the screw or fastener to pivot relative to the center hub 210 (e.g., the hole or opening may be configured as a variable angled screw hole). For example, the hole or opening may include a plurality of fins arranged and configured to enable the screw fastener to be angulated relative to the center hub 210 of the bone plate 200. That is, for example, the hole or opening could include fins and/or slots to allow for multiple positions at various angles. As such, the bone plate 200 should not be limited to any number, arrangement, or configuration of holes or openings unless explicitly claimed.

As illustrated in FIG. 5, the bone plate 200 may include a plurality of arms 230 extending from the center hub 210. For example, as illustrated, the bone plate 200 may include six arms 230 extending from the center hub 210. In the illustrated example, the bone plate 200 may include first and second arms 230a, 230b extending superiorly from the center hub 210. In one exemplary use case, the first and second superiorly extending arms 230a, 230b may be used to wrap or bend around a proximal portion of a bone fragment to prevent it from moving superiorly/inferiorly, as will be shown and described in greater detail below. In addition, as illustrated, the bone plate 200 may include third and fourth arms 230c, 230d extending laterally from the center hub 210 and fifth and sixth arms 232e, 232f extending medially from the center hub 210. In use, the third, fourth, fifth, and sixth arms 232c-232f may be used to wrap or bend around a portion of retained anatomy as needed.

As illustrated in FIG. 5, each arm 230 may include a plurality of holes or openings 232 for receiving a screw or fastener to secure the arms 230 to, for example, a patient's bone. It should be appreciated that while each arm 230 is shown with three holes or openings, the arms 230 may include any number of holes or openings provided in any arrangement or configuration including, for example, one, two, four, five, or more holes or openings. Moreover, while a particular example of a hole or opening 232 is shown, it should be appreciated that the hole or opening 232 may have any now known or hereafter developed configuration. For example, the hole or opening 232 may include threads for threadably engaging a head portion of the screw or fastener (e.g., hole or opening may be configured as a locking screw opening). Alternatively, the hole or opening 232 may be arranged and configured to enable the screw or fastener to pivot relative to the arms 230 (e.g., the hole or opening may be configured as a variable angled screw hole). For example, the hole or opening 232 may include a plurality of fins arranged and configured to enable the screw fastener to be angulated relative to the arms of the bone plate 200. That is, for example, the hole or opening could include fins and/or slots to allow for multiple positions at various angles. As such, the bone plate 200 should not be limited to any number, arrangement, or configuration of holes or openings 232 unless explicitly claimed.

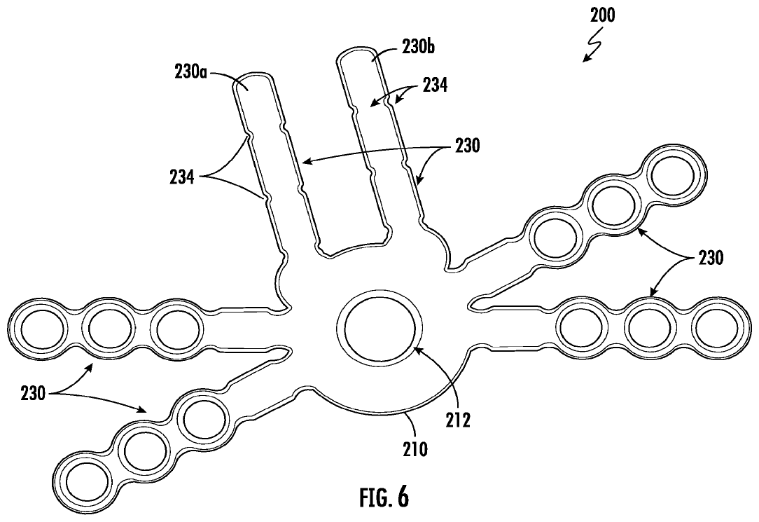

Alternatively, as illustrated in FIG. 6, one or more of the arms 230 (shown as superiorly extending arms 230a, 230b) may be completely devoid of any holes or openings. In some examples, as illustrated, the arms 230 (shown as superiorly extending arms 230a, 230b) may be arranged and configured with one or more weakened regions 234 that facilitate removal and/or bending of the arms 230. Thus arranged, for example, the arms 230 can be cut and/or adjusted as needed.

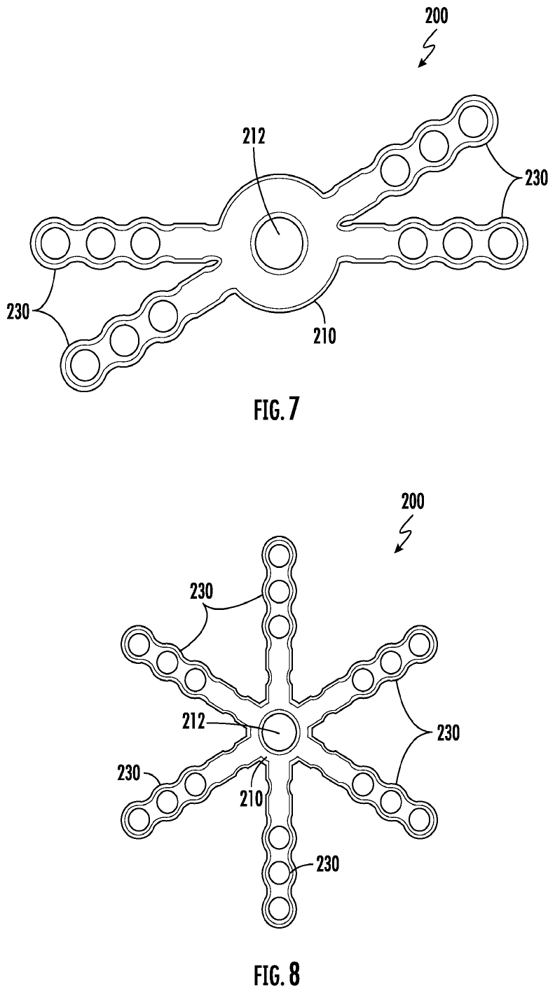

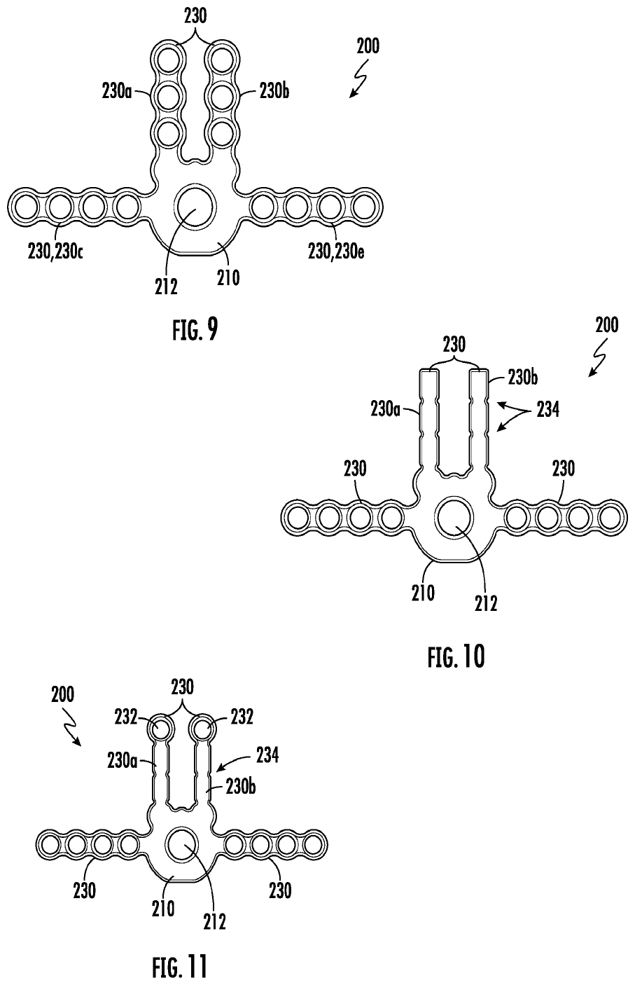

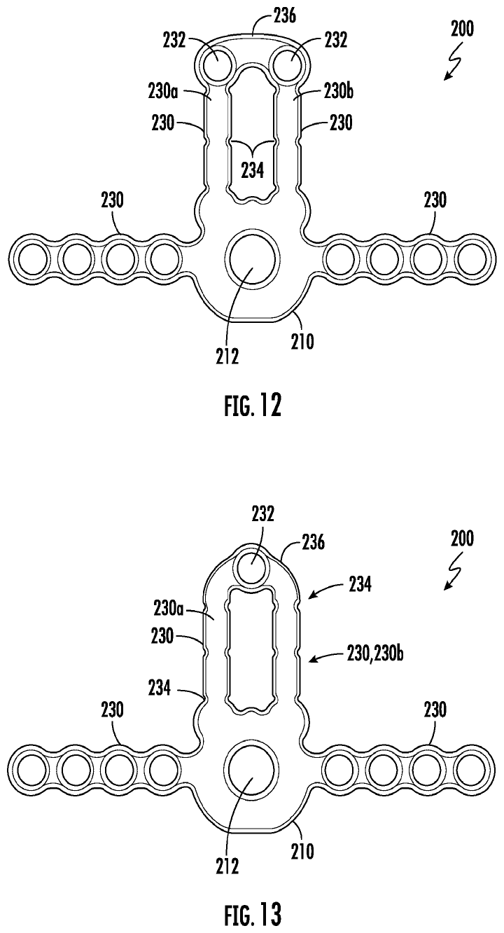

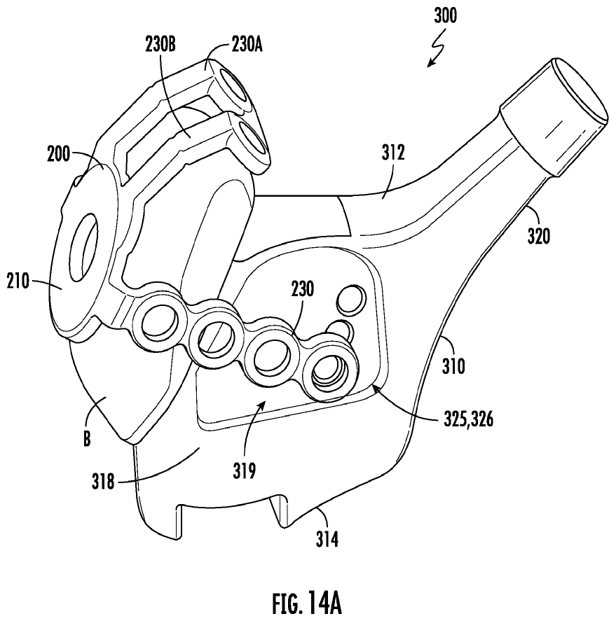

While particular examples of the bone plate 200 in accordance with one or more features of the present disclosure have been shown and described in connection with FIGS. 5 and 6, it should be appreciated that the bone plates 200 can be provided in any number of variations and/or configurations. For example, FIGS. 7-13 illustrate various alternate examples of bone plates 200 in accordance with one or more features of the present disclosure. For example, with reference to FIG. 7, the bone plate 200 may include a center hub 210 and laterally extending arms 230 without any superiorly extending arms. With reference to FIG. 8, the bone plate 200 may include a center hub 210 and a plurality of circumferentially extending arms 230. With reference to FIG. 9, the bone plate 200 may include a center hub 210 with first and second superiorly extending arms 230a, 230b and only one medial and lateral extending arms 230c, 230e. With reference to FIG. 10, the first and second superiorly extending arms 230a, 230b may be devoid of any holes or openings, the first and second superiorly extending arms 230a, 230b including weakened regions 234 to facilitate cutting and/or bending. With reference to FIG. 11, the first and second superiorly extending arms 230a, 230b may each include weakened regions 234 to facilitate cutting and/or bending and a hole or opening 232 formed at an end thereof. With reference to FIG. 12, the first and second superiorly extending arms 230a, 230b may each include weakened regions 234 to facilitate cutting and/or bending and a bridge 236 at an end thereof. The bridge 236 coupling the first arm 230a to the second arm 230b. As illustrated, the bridge 236 may include first and second holes or openings 232 formed therein. With reference to FIG. 13, the bridge 236 may include a single hole or opening 232 formed therein.

As should be appreciated the number of alternate examples is endless. In use, in accordance with one or more features of the present disclosure, the bone plates provide surgeons with increased flexibility in customizing a solution for each individual patient.

### Proximal Femur Replacement Implant Assembly

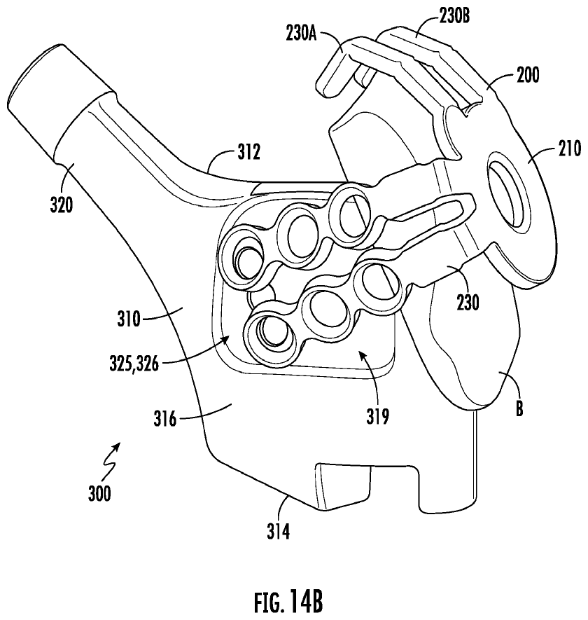

In accordance with one or more features of the present disclosure, the bone plate 200 such as, for example, bone plate 200 shown in FIG. 12 (FIG. 14A) or bone plate 200 shown in FIG. 6 (FIG. 14B), is well suited for use in a proximal femur replacement procedure. That is, with reference to FIGS. 14A and 14B, in some assembly and/or methods of use, the bone plate 200 can be used in combination with a proximal femur replacement or greater trochanter implant assembly 300 (terms used interchangeably herein).

That is, in use, a proximal femur replacement implant assembly 300 includes a proximal femur replacement implant 310 arranged and configured to replace, or at least partially replace, the head portion or proximal end of a patient's femur. The proximal femur replacement implant 310 may be arranged and configured to be implanted at least partially within an intramedullary canal of the patient's femur (e.g., the proximal femur replacement implant 310 may include a stem portion arranged and configured to be inserted into the intramedullary canal of the patient's femur). In addition, in accordance with one or more features of the present disclosure, the proximal femur replacement implant assembly 300 includes a customizable bone plate 200 arranged and configured to be coupled to the patient's bone and/or soft tissue. In addition, the customizable bone plate 200 is arranged and configured to couple to the proximal femur replacement implant 310. In use, the customizable bone plate 200 enables increased flexibility in positioning of the bone plate 200 relative to the proximal femur replacement implant 310 thus enabling a surgeon to better tailor the assembly 300 to individual patient's needs. In addition, since the customizable bone plate 200 may be arranged and configured to couple to the proximal femur replacement implant 310 on either side thereof, the assembly 300 can be used on either the left or right proximal femur thus eliminating the need to manufacture and provide handed proximal femur replacement implants 310. Thus arranged, the greater trochanter may be reattached to the proximal femur replacement implant 310 with sufficient constraint to allow for porous ingrowth and overall system longevity.

With reference to FIGS. 14A and 14B, and as previously mentioned, the proximal femur replacement implant assembly 300 includes a proximal femur replacement implant 310 and a customizable bone plate 200 arranged and configured to couple the patient's bone to the proximal femur replacement implant 310.

As illustrated, the proximal femur replacement implant 310 includes a proximal end 312, a distal end 314, a first side surface 316, and a second side surface 318. In use, the proximal femur replacement implant 310 includes a neck 320 extending from the proximal end 312. In use, as will be appreciated by one of ordinary skill in the art, the neck 320 is arranged and configured to receive a femoral head configured to interact with an acetabular system and/or hip socket. In addition, and as will be appreciated by one of ordinary skill in the art, the proximal femur replacement implant 310 includes an intramedullary stem arranged and configured to be inserted into an intramedullary canal of a patient's bone (e.g., femur). As will be appreciated by one of ordinary skill in the art, in use, the patient's femur is initially prepared for implantation of the proximal femur replacement implant 310 by, for example, resecting the proximal femur and reaming the intramedullary canal in the patient's femur. The stem of the proximal femur replacement implant 310 is then inserted into the intramedullary canal of the resected femur.

In accordance with one or more features of the present disclosure, the proximal femur replacement implant 310 includes an attachment mechanism 325 arranged and configured to couple the customizable bone plate 200 to the proximal femur replacement implant 310. In use, the attachment mechanism 325 may be any suitable mechanism now known or hereafter developed. For example, as illustrated, the attachment mechanism 325 may be provided in the form of one or more screw fixation holes 326 formed in the first and second side surfaces 316, 318. In use, and as will be described in greater detail below, the one or more screw fixation holes 326 are arranged and configured to receive one or more fasteners, respectively, for coupling the customizable bone plate 200 to the first and second side surfaces 316, 318 of the proximal femur replacement implant 310.

As illustrated, in some examples, the proximal femur replacement implant 310 may include a plurality of screw fixation holes 326 such as, for example, three screw fixation holes. However, it should be appreciated that any number and arrangement of screw fixation holes may be provided including, more or less screw fixation holes arranged in any arrangement. In addition, while the screw fixation holes are shown as being internally threaded, it should be appreciated that the screw fixation holes may be provided in any now known or hereafter developed configuration including, for example, including a plurality of fins arranged and configured to enable the screw fastener to be angulated relative to the proximal femur replacement implant 310. That is, one or more of the screw fixation holes could include fins and/or slots to allow for multiple positions at various angles.

As illustrated, in some examples, the first and second side surfaces 316, 318 of the proximal femur replacement implant 310 may each include a recessed surface 319 with the plurality of screw fixation holes 326 positioned within the recessed surfaces 319. In use, the recessed surface 319 may be the same on either side 316, 318 of the implant 310 to prevent the need for handed implants. Thus arranged, the recessed surface 319 may be arranged and configured to be recessed a sufficient distance to prevent the bone plate 200 and/or fasteners from extending a greater distance beyond an outer surface of the non-recessed surface of the implant 310 thereby minimizing associated patient trauma (e.g., the recessed surfaces 319 reduce the extent or distance that the customizable bone plate 200 and/or fasteners extend from the outer surface of the proximal femur replacement implant 310 to minimize associated patient trauma).

It should be appreciated while a particular proximal femur replacement implant 310 is shown and described, it should be understood that any proximal femur replacement implant 310 now known or hereafter described can be used including, for example, it is envisioned that a conventional femoral stem may be used in place of the proximal femur replacement implant. As such, the present disclosure should not be limited to any particular proximal femur replacement implant 310. Thus, for the sake of brevity, additional detailed discussion on the proximal femur replacement implant 310 is omitted herefrom.

As illustrated, in use, the surgeon may position the center hub 210 of the bone plate 200 adjacent to the patient's bone B. Thereafter, the superiorly extending arms 230a, 230b may be bent and wrapped about the superior portion of the patient's bone B. In use, the bone plate 200 secures, holds, or the like, the bone B securely enough against the implant to promote bone ingrowth into the proximal femur replacement implant 310. In addition, by preserving the patient's bone B, the soft tissue connection of the bone B is maintained, which typically provides a significantly stronger connection than if the soft tissue was detached from the bone B and was then subsequently sutured to the proximal femur replacement implant 310. The remaining arms 230 can be wrapped medially and laterally to encompass the patient's bone B and to couple to either side 316, 318 of the proximal femur replacement implant 310. In addition, any remaining arms 230 can be utilized or removed as desired. Thus arranged, the bone plate 200, and more specifically, the arms 230 of the bone plate 200, can be customized (e.g., bent) around the bone fragment B to secure it appropriately. In some examples, as mentioned elsewhere herein, the bone contacting surfaces of the implant and bone plates may include a porous surface for enabling bone ingrowth with the patient's bone. In addition, and/or alternatively, the bone contacting surfaces may include roughening or a roughened surface to promote bone ingrowth.

Thus arranged, in use, the customizable bone plates 200 provide surgeons a more secure mechanical fixation to adhere the bone patient anatomy to the proximal femur replacement implant 310. Thus, providing improved coupling of the components and specifically to securement of the patient's bone B to the porous coating on the implant 310 to provide improve bone-ingrowth.

### Proximal Tibial Replacement Implant Assembly

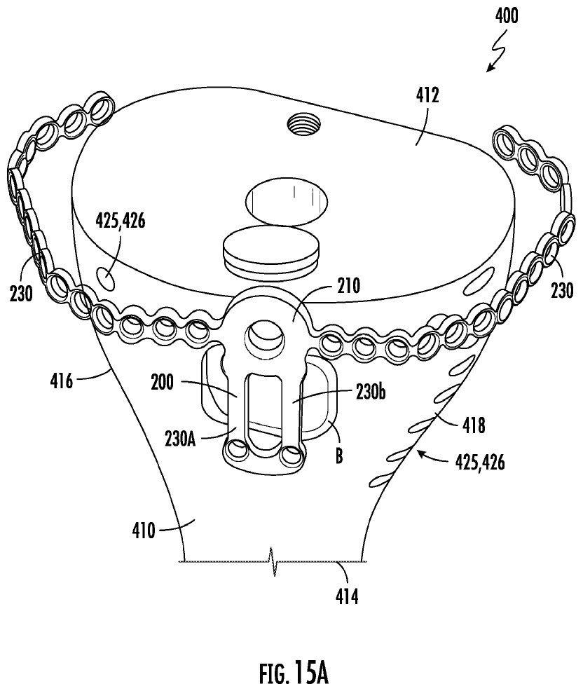

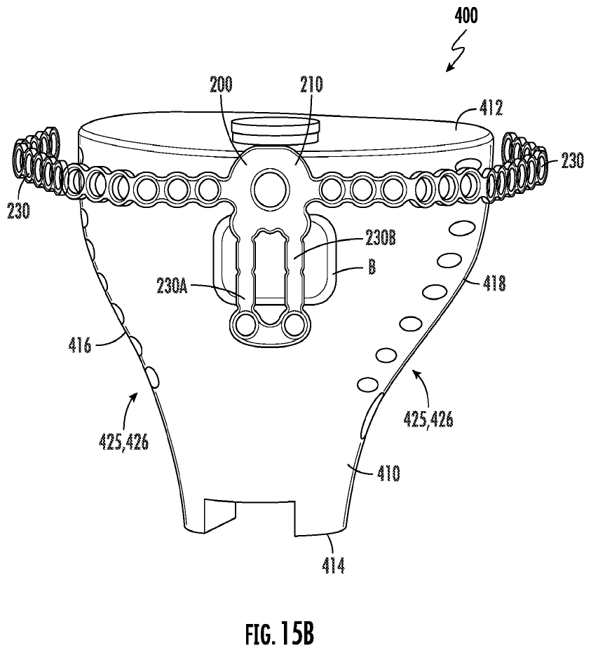

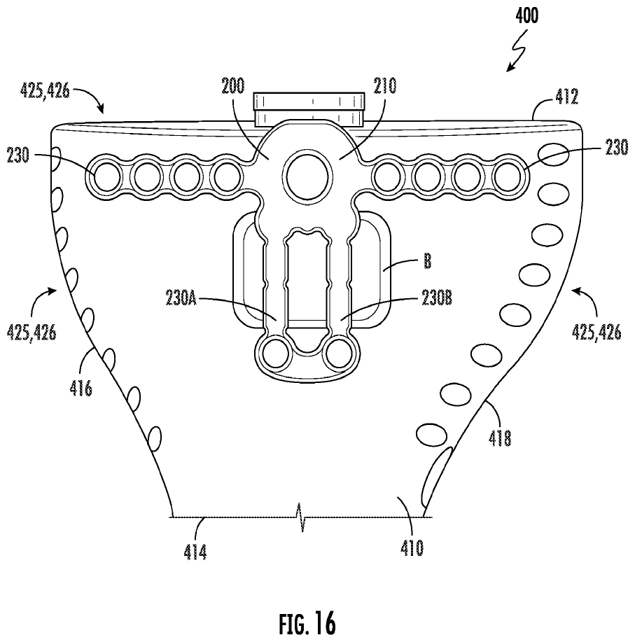

In accordance with one or more features of the present disclosure, the bone plate 200 such as, for example, bone plate 200 shown in FIG. 12, is well suited for use in a proximal tibial replacement procedure. That is, with reference to FIGS. 15A, 15B, and 16, in some methods of use, the bone plate 200 can be used in combination with a proximal tibia replacement implant assembly 400.

That is, in use, a proximal tibia replacement implant assembly 400 includes a proximal tibia replacement implant 410 arranged and configured to replace, or at least partially replace, the head portion or proximal end of a patient's tibia. The proximal tibia replacement implant 410 may be arranged and configured to be implanted within an intramedullary canal of the patient's tibia (e.g., the proximal tibia replacement implant 410 may include a stem portion arranged and configured to be inserted into the intramedullary canal of the patient's tibia). In addition, in accordance with one or more features of the present disclosure, the proximal tibia replacement implant assembly 400 includes a customizable bone plate 200 arranged and configured to be coupled to the patient's bone B and/or soft tissue. In addition, the customizable bone plate 200 is arranged and configured to couple to the proximal tibia replacement implant 410. In use, the customizable bone plate 200 enables increased flexibility in positioning of the bone plate 200 relative to the proximal tibia replacement implant 410 thus enabling a surgeon to better tailor the assembly 400 to individual patient's needs. Thus arranged, the customizable bone plate 200 can be coupled to the proximal tibia replacement implant 410 with sufficient constraint to allow for porous ingrowth and overall system longevity.

With reference to FIGS. 15A, 15B, and 16, and as previously mentioned, the proximal tibia replacement implant assembly 400 includes a proximal tibia replacement implant 410 and a customizable bone plate 200 arranged and configured to couple the patient's bone B to the proximal tibia replacement implant 410. In some examples, while a single customizable bone plate 200 is shown and described, this is but one configuration and alternate configurations are envisioned including incorporation of multiple bone plates.

As illustrated, the proximal tibia replacement implant 410 includes a proximal end 412, a distal end 414, a first side surface 416, and a second side surface 418. The proximal end 412 of the proximal tibia replacement implant 410 defines a top surface arranged and configured to receive, for example, an articular insert or other opposing surface(s) and/or hinged knee linkage(s) from a distal femoral implant and/or knee implant. For example, the top surface of the proximal tibia replacement implant 410 may include a connection mechanism (e.g., illustrated as a hole) for receiving, coupling, etc. to, for example, a tibial tray, a hinged knee arthroplasty, etc. In use, the connection mechanism can vary, but all mechanisms preferably have the ability to enable translation (proximal/distal to the tibia axis) to connect the femoral implant to the tibial implant. In addition, and as will be appreciated by one of ordinary skill in the art, the proximal tibia replacement implant 410 includes an intramedullary stem arranged and configured to be inserted into an intramedullary canal of a patient's bone (e.g., tibia). As will be appreciated by one of ordinary skill in the art, in use, the patient's tibia is initially prepared for implantation of the proximal tibia replacement implant 410 by, for example, resecting the proximal tibia and reaming the intramedullary canal in the patient's tibia. The stem of the proximal tibia replacement implant 410 is then inserted into the intramedullary canal of the resected tibia.

In accordance with one or more features of the present disclosure, the proximal tibia replacement implant 410 includes an attachment mechanism 425 arranged and configured to couple the customizable bone plate 200 to the proximal tibia replacement implant 410. In use, the attachment mechanism 425 may be any suitable mechanism now known or hereafter developed. For example, as illustrated, the attachment mechanism 425 may be provided in the form of one or more screw fixation holes 426 formed in the surfaces of the proximal tibia replacement implant 410. In use, and as will be described in greater detail below, the one or more screw fixation holes 426 are arranged and configured to receive one or more fasteners, respectively, for coupling the customizable bone plate 200 to the proximal tibia replacement implant 410.

As illustrated, in some examples, the proximal tibia replacement implant 410 may include a plurality of screw fixation holes 426. In use, it should be appreciated that any number and arrangement of screw fixation holes may be provided. In addition, while the screw fixation holes are shown as being internally threaded, it should be appreciated that the screw fixation holes may be provided in any now known or hereafter developed configuration including, for example, including a plurality of fins arranged and configured to enable the screw fastener to be angulated relative to the proximal tibia replacement implant 410. That is, one or more of the screw fixation holes could include fins and/or slots to allow for multiple positions at various angles.

It should be appreciated while a particular proximal tibia replacement implant 410 is shown and described, it should be understood that any proximal tibia replacement implant 410 now known or hereafter described can be used. As such, the present disclosure should not be limited to any particular proximal tibia replacement implant 410. Thus, for the sake of brevity, additional detailed discussion on the proximal tibia replacement implant 410 is omitted herefrom.

As illustrated, in use, the surgeon may position the center hub 210 of the bone plate 200 adjacent to the patient's bone B. Thereafter, the customizable bone plate 200 may be positioned so that the superiorly extending arms 230a, 230b may be positioned downwards (e.g., customizable bone plate 200 may be positioned upside down so that the superior arms extend inferiorly). Next, the remaining arms 230 (e.g., medial and lateral arms) may be positioned so that they wrap around, or at least partially about, the proximal tibia replacement implant 410. Any remaining arms can be utilized or removed as desired. Thus arranged, as illustrated, the customizable bone plate 200 can be used to wrap about and/or encompass the patient's bone B and to couple to the proximal tibia replacement implant 410. As such, the bone plate, and more specifically, the arms of the bone plate, can be customized (e.g., bent) around the bone fragment to secure it appropriately.

Thus arranged, in use, the customizable bone plates 200 provide surgeons a more secure mechanical fixation to adhere the bone patient anatomy to the proximal tibia replacement implant 410. Thus, providing improved coupling of the components and specifically to securement of the patient's bone to the porous coating on the implant to provide improve bone-ingrowth.

In use, the customizable bone plates can be used with or without additional trauma plates while in combination with the proximal femur and proximal tibia replacements.

In accordance with features of the present disclosure, by utilizing a customizable bone plate surgeons are provided with the ability to tailor the assemblies as needed to, for example, fix any remaining bone and/or soft tissue (e.g., provides increased flexibility in determining proper fixation positioning). For example, variation in height or position of the bone plate relative to the proximal femur replacement implant or proximal tibial replacement implant can be accommodated thus providing surgeons with increased flexibility to reposition the bone plate with respect to the proximal femur replacement implant or proximal tibia replacement implant. In addition, the assemblies prevent, or at least minimize, superior/inferior and anterior/posterior movements of the bone B. That is, utilization of the bone plate enables a surgeon to reposition the patient's remaining bone B in the superior-inferior direction and the medial-lateral direction, then once the bone B is properly positioned, the surgeon can secure the position of the various components, and hence the patient's bone B and/or soft tissue. Thus arranged, the assemblies can limit anterior/posterior translation while retaining proximal/distal and medial/lateral constraints, while also minimizing micromotion to better accommodate porous ingrowth between the preserved bone B and surgical implants.

### Proximal Femur Replacement Implant Assembly

As previously mentioned herein, in conventional methods of use, reattaching bone such as, for example, the patient's greater trochanter to a proximal femur replacement implant assembly is usually accomplished via attachment mechanisms typically consisting of sutures as a ligature mechanism. Alternatively, and/or in addition, as previously mentioned and as illustrated herein, the bone B may be a piece or portion of a patient's bone such as an intact piece of bone or a chipped bone fragment. Alternatively, the bone B may be a piece of allograft, bone slurry, bone putty, etc. One disadvantage of this approach, however, is that the typical extent of micromotion experienced from this type of attachment is greater than what is typically desired for enabling porous ingrowth between the bone B and the implant. Additionally, sutures used in this type of surgical procedure are typically less capable of constraining the bone B (e.g., patient's greater trochanter) from anterior/posterior translation.

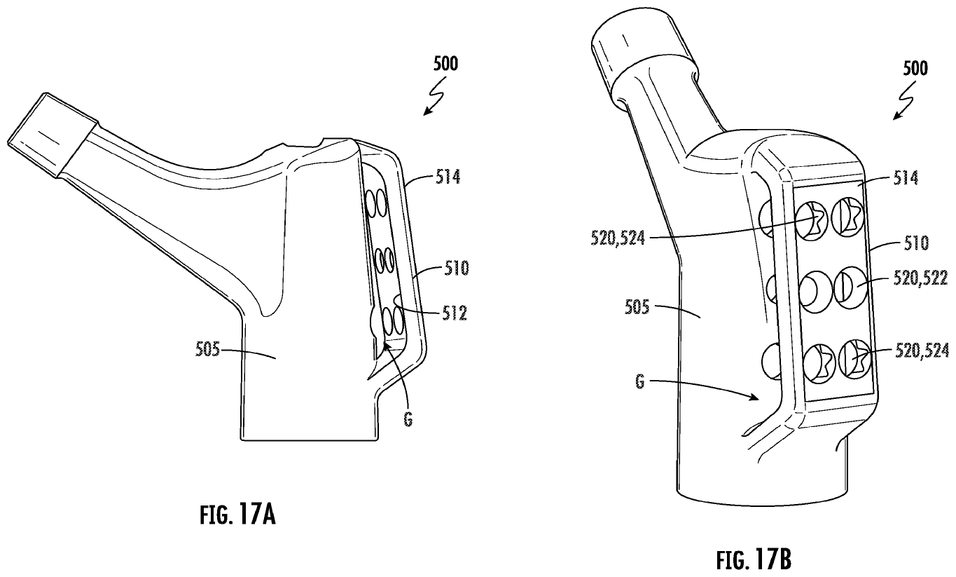

To overcome this disadvantage, with reference to FIGS. 17A-17C, an alternate example of a proximal femur replacement implant assembly 500 in accordance with one or more features of the present disclosure is shown. As illustrated, the proximal femur replacement implant assembly 500 includes a proximal femur replacement implant 505 and a bridge member 510 (e.g., a bone plate or portion of a bone plate) integrally and monolithically formed with the proximal femur replacement implant 505, although this is but one configuration and the bridge member 510 may be separately formed and coupled thereto. In use, as illustrated, the bridge member 510 extends laterally from the proximal femur replacement implant 505. Thus arranged, the inner surface of the bridge member 510 and the outer surface of the proximal femur replacement implant 505 such as, for example, the outer surface of the lateral surface or portion of the proximal femur replacement implant 505, define a space or a gap G. In various examples, the surfaces of the proximal femur replacement implant 505 including, without limitation, the outer surface thereof along with the surfaces (e.g., inner and outer surfaces) of the bridge member 510 may include a porous coating or a plurality of porous coatings, if the bone B is slotted to fit within the implant (as will be described in greater detail below). In use, by providing a plurality of porous coatings, the implant can be configured to promote bone ingrowth from either or both sides of the implant. Thus arranged, as will be described in greater detail below, in use, the gap G is arranged and configured to receive a portion of the bone B with the bone B in contact with the porous coatings of the proximal femur replacement implant 505.

More specifically, as illustrated, the bridge member 510 includes an inner or implant facing surface 512 and an outer surface 514. Moreover, the bridge member 510 includes a plurality of openings 520 formed therein for receiving a plurality of fasteners or screws (terms used interchangeably herein without the intent to limit or distinguish) for reasons that will be described below.

As will be appreciated by one of ordinary skill in the art, the openings 520 may be in the form of a locking screw opening 522 or a variable angled opening 524. Locking screw openings 522 generally include a plurality of threads formed on an inner surface thereof for mating with threads formed on an outer surface of a head portion of a fastener. Thus arranged, the fastener may be said to be locked to the implant (e.g., bridge member 510) via the locking screw openings 522. That is, as will be appreciated by one of ordinary skill in the art, the fastener is threaded into one of the locking screw openings 522 formed in the bridge member 510. The fastener is secured to the bridge member 510 via threads formed on the head portion of the fastener that cooperate with the threaded locking screw opening 522 formed in the bridge member 510. This provides rigid fixation between the bridge member 510 and the fasteners.

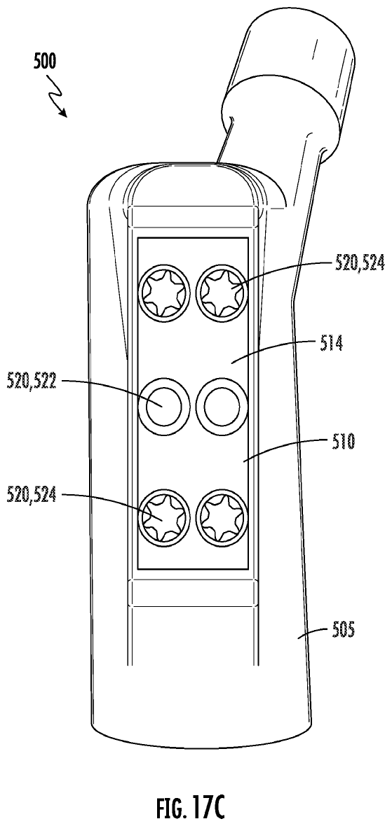

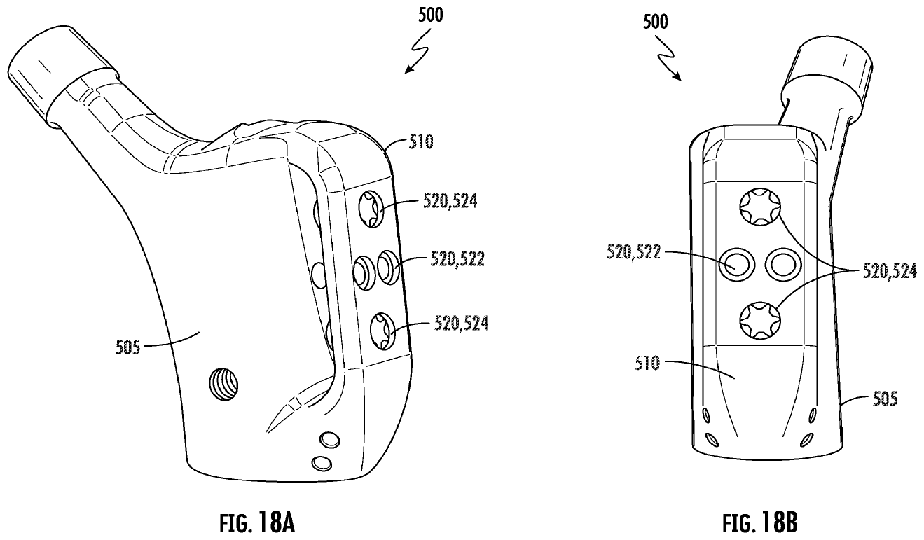

In addition, with additional reference to FIGS. 18A and 18B, the bridge member 510 may also include a plurality of variable angled openings 524 formed therein for receiving a non-locking or variable angled (e.g., polyaxial) fastener. In use, the variable angled openings 524 are arranged and configured to enable the fastener inserted therein to achieve a greater range of insertion angles as compared to, for example, a conventional locking screw that is threadably coupled to the bridge member 510. For example, the angular position of the fastener may be rotated through a range of approximately ±15 degrees, although the range of allowable polyaxial rotation can vary, including greater and less than the fifteen degrees. In use, the variable angled openings 524 may be provided in any suitable manner, configuration, etc. now known or hereafter developed for enabling polyaxial positioning or angling of the fastener relative to the bridge member 510.

In some examples, as illustrated in FIGS. 18A and 18B, the variable angled openings 524 may include fins or projections that extend radially inward from an inner surface of the openings and into an interior region of the openings. In normal use, the fins are configured to engage or cooperate with the head portion of the fastener. In use, the fins engage the head portion of the fastener in order to secure the fastener at a desired position and at a desired angular orientation within the variable angled opening 524. Additional information on the operation and configuration of the fins can be found in U.S. patent application Ser. No. 15/706,877, with an earliest filing date of Jul. 25, 2005, now U.S. Pat. No. 10,092,337 entitled “Systems and Methods for Using Polyaxial Plates”; U.S. patent application Ser. No. 13/524,506, filed on Jun. 15, 2012, entitled “Variable Angle Locking Implant”, and International PCT Patent Application No. WO20200247381, filed on Jun. 2, 2020, entitled “Orthopedic Implant with Improved Variable Angle Locking Mechanism”, the entire contents of which are hereby incorporated by reference.

It should be appreciated that the bridge members 510 illustrated in FIGS. 17A-18B are but some configurations and the bridge members may be configured to have any number of openings in any configuration of locking screw openings and variable angled openings.

In some examples, as previously mentioned, the bone contacting surfaces of the proximal femur replacement implant 505 and/or the bone contacting surfaces of the bridge member 510 include one or more porous surfaces, in addition to the plurality of opening 520. In use, the plurality of openings 520 may be used to couple the bone B to the proximal femur replacement implant 505. For example, the plurality of openings 520 may serve as attachment points between the proximal femur replacement implant 505 and a supplemental trauma device such as, for example, a bone plate such as, for example, a hook plate 132 as previously described (e.g., bridge member 510 eliminates the necessity for incorporation of a secondary bone plate as previously described). Thus arranged, a bone plate such as, for example, the hook plate 132, can be used in conjunction with the proximal femur replacement implant assembly 500 to better secure the bone B (e.g., patient's greater trochanter) to the implant's porous surface. In use, fasteners can be used to couple the bone plate to the proximal femur replacement implant 505 via the plurality of openings 520. In some examples, the design enables 0 to 20 degrees of version built into its designs in 5 degree increments to accommodate the version from the acetabular cup.

Alternatively, and/or in addition, the plurality of openings 520 may serve as attachment points between the bone B and the proximal femur replacement implant 505, thereby enabling a surgeon to directly couple the bone B to the proximal femur replacement implant 505 utilizing one or more fasteners and/or sutures, ligaments, cables, etc. as will be described in greater detail below.

In use, the geometry of the proximal femur replacement implant 505 provides surgeons with increased flexibility to facilitate coupling of bone B such as, for example, a patient's greater trochanter to the proximal femur replacement implant 505. In use, the surgeon can elect to couple one or more bone plates to the proximal femur replacement implant 505 with, for example, the bone B positioned between the bridge member 510 and the bone plate and/or with the bone B positioned between the proximal femur replacement implant 505 and the bridge member 510. In use, the bone plates can be connected to the bridge member 510 via one or more bone fasteners such as, for example, locking screws, variable angle locking screws, or the like. Alternatively, and/or in addition, the surgeon can elect to directly couple the bone B to the proximal femur replacement implant 505 via one or more fasteners and/or one or more sutures, ligaments, cables, etc. That is, the surgeon can elect to utilize one or more fixation members incorporating an aperture for a synthetic ligament, suture, cable, etc., as will be described in greater detail below.

Thus arranged, the bone B such as, for example, the patient's greater trochanter, can be reattached to the proximal femur replacement implant 505 with sufficient constraint to allow for porous ingrowth and overall system longevity. In use, the proximal femur replacement implant assembly 500 is configured to limit anterior/posterior and/or medial/lateral translation in a manner that retains the proximal/distal and medial/lateral constraints, while also minimizing micromotion to better accommodate porous ingrowth between the bone B and the replacement implant. In some examples, the attachment mechanism allows multiple utility by including screw holes, variable angle screw holes, a slot, etc. to enable a synthetic ligament, suture, cable, etc. if limited or no bone is able to be preserved. Moreover, by incorporating a variety of screw angles and a number of screws, surgeons are provided with increased versatility to facilitate the best compatibility with what bone can be preserved during the operation. In comparison to prior art designs, the proximal femur replacement implant assembly 500 possesses enhanced performance, enhanced clinical benefits, enhanced safety, and increased system longevity.

### Cable Saddle Fasteners or Screws

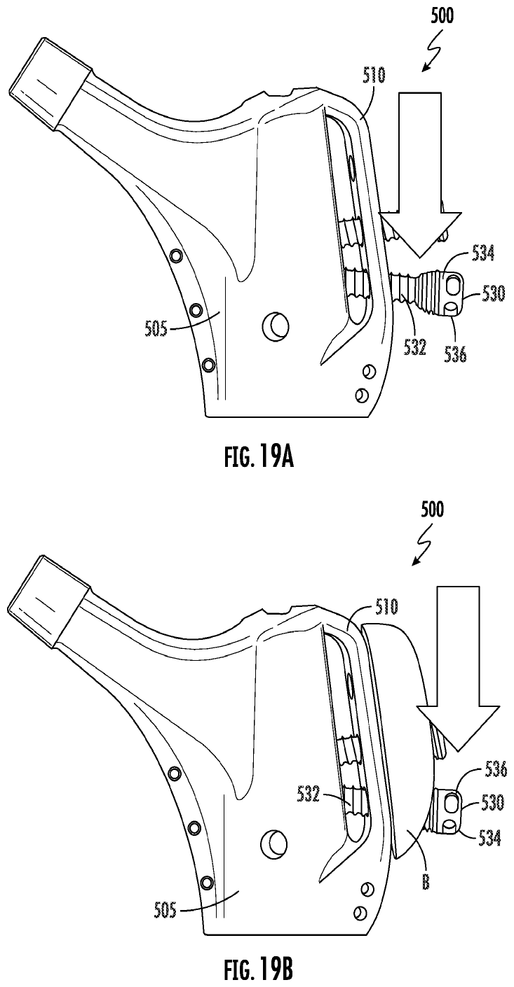

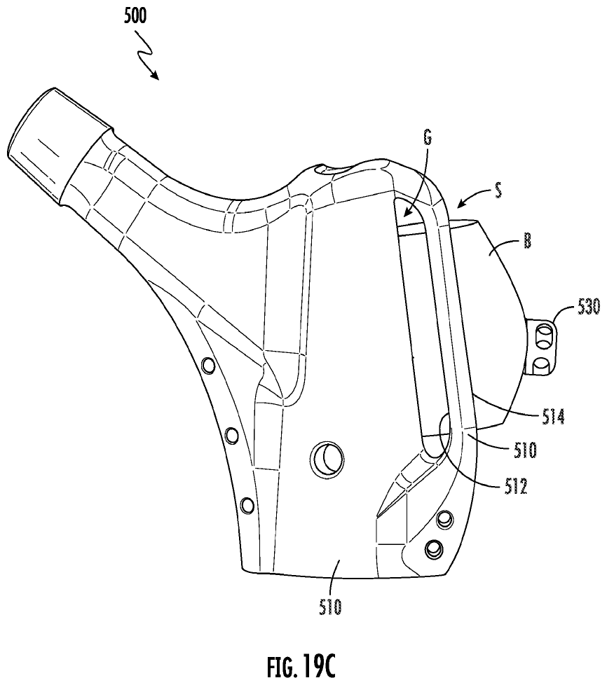

With additional reference to FIGS. 19A and 19B, a cable saddle screw 530 may be used. As previously mentioned, in use, the cable saddle screws 530 may be used to secure a ligament, suture, cable, etc. such as, for example, a synthetic ligament. That is, as illustrated, the cable saddle screw 530 includes a threaded shaft 532 and an enlarged head portion 534 including a plurality of transverse openings 536 formed therein, the openings 536 arranged and configured to enable a ligament, suture, cable, etc. (terms used interchangeably herein without the intent to limit or distinguish) to pass therethrough. In use, the openings 520 formed in the bridge member 510 of the proximal femur replacement implant 505 may be arranged and configured with variable locking tabs that have been modified to work with, engage, etc. the threaded shaft 532 of the cable saddle screws 530 as opposed to the head portion 534.

That is, the geometry of the fins in the variable angled openings 524 of the bridge member 510 are arranged and configured to interact, engage, etc. with the threaded shaft portion of the fastener (e.g., as opposed to the head portion of the fastener). As such, the fins are configured to enable the external threads formed on the threaded shaft 532 of the fastener 530 to engage the threaded shaft 532 at various angles up to 15 degrees and/or to deform to securely couple the threaded shaft 532 to the bridge member 510. As such, in some examples, the proximal femur replacement implant 505 includes a plurality of variable angled openings 524 that include a plurality of tabs or fins that are configured to interact with the threaded shaft 532 of a corresponding fastener such as, for example, a cable saddle screw 530, thereby allowing the ability to retain or project the head portion of the fastener externally from the bone B. Thus arranged, features such as, for example, the transverse openings 536, can be incorporated into the head 534 of the fastener 530 to better address the problem of soft tissue re-attachment and/or bone attachment.

That is, the cable saddle screws 530 can be coupled to the bridge member 510 of the proximal femur replacement implant 505 such that the enlarged head portion 534 extends externally from the bone B enabling surgeons to utilize the plurality of transverse openings 536 formed in the head portion 534 of the cable saddle screws 530 to facilitate soft tissue re-attachment and/or bone attachment, although this is but one configuration and other configurations are envisioned such as, for example, using slots or other geometries in the head portion to retain a cable or suture.

In use, by positioning the cable saddle (e.g., transverse openings 536) at the end of the screw (e.g., the enlarged head portion 534 at the end of the threaded shaft 532), the cable saddle screws 530 enable a ligament to be wrapped around the bone B or soft tissue and to be inserted, pass through, etc. the enlarged head portion 534 of the cable saddle screw 530, and prevent the ligament from migrating over time. As such, cable saddle screws 530 may be utilized in connection with the proximal femur replacement implant assembly 500 to address, or minimize, micromotion by limiting anterior/posterior and/or medial/lateral translation in a way that retains the proximal/distal and medial/lateral constraints, while also minimizing micromotion to better accommodate porous ingrowth between the preserved bone and the orthopedic implant. This attachment mechanism allows significant versatility by being able to screw into various holes 520 of an implant at various angles and locations on the bone B to better preserve any bone fragment that can be retained. Alternatively, and/or in addition, in use, the ligament may be passed directly through one or more of the holes 520 formed in the implant.

Thus arranged, by utilizing one or more cable saddle screws (e.g., by integrating a cable saddle head portion with a threaded shaft portion of a screw), a surgeon can connect one or more cables, ligaments, sutures, etc. to the proximal femur replacement implant 505 via the cable saddle screws 530 and bridge member 510. In use, this combination prevents the cables, ligaments, sutures, etc. from migrating while simultaneously having screw fixation in the same location physically connecting the retained bone or soft tissue to the implant itself. Thereby allowing for a wide range of versatility when reattaching bone with soft tissue. As such, in use, for example, the patient's greater trochanter can be reattached to the proximal femur replacement implant assembly 500 with sufficient constraint to allow for porous ingrowth and overall system longevity. This concept can be carried out with a variety of screw angles and number of screws to allow the best compatibility with what bone can be preserved during the operation.

With reference to FIG. 19C, in an exemplary method of use, a patient's greater trochanter can be cut to form a groove or slot S therein. Thereafter, the patient's greater trochanter can be positioned so that the slot S formed therein aligns with the bridge member 510. Thereafter, by inserting, pushing, etc. the patient's greater trochanter, a first portion of the patient's greater trochanter may be positioned within the gap G positioned between the outer surface of the proximal femur replacement implant 505 and the inner surface 512 of the bridge member 510 while a second portion of the proximal femur replacement implant 505 remains in contact with the outer surface 514 of the bridge member 514. Thus arranged, improved securement of the greater trochanter is provided (e.g., improved initial fixation of the greater trochanter can be achieved).

In addition, and/or alternatively, as illustrated, in some examples, one or more fasteners such as, for example, cable saddled screws 530, can be subsequently utilized to provide additional fixation of the patient's greater trochanter to the proximal femur replacement implant 505 via the fasteners directly and/or via utilization of one or more ligaments.

### Initial Fixation

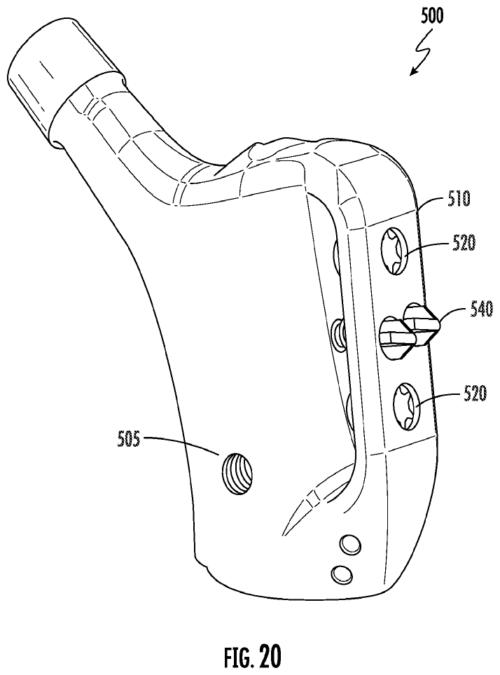

With reference to FIG. 20, in accordance with one or more features of the present disclosure that can be utilized in combination and/or separately from the other features disclosed herein, the bridge member 510 may include one or more spikes or projections 540 extending therefrom for facilitating initial fixation to the bone B to reduce micromotion during initial implantation to facilitate improved porous ingrowth. In use, the one or more spikes 540 may be used to provide initial fixation to the bone B or to an augment, which can be attached to the proximal femur replacement implant 505.

In use, in some examples, the one or more spikes 540 can be used in combination with one or more fasteners in order to provide both spiked fixation and screw fixation, although this is but one configuration and other configurations are envisioned such as, for example, a stand-alone augment utilizing one or more cables, ligaments, sutures, etc. to preserve bone and soft tissue thereby allowing a wide range of versatility when reattaching bone with soft tissue.

As illustrated, in some examples, the spikes 540 can be attached to the proximal femur replacement implant 505 through a threaded mechanism. For example, as illustrated, in some examples, the spikes 540 may include a threaded shaft which is arranged and configured to engage one of the holes formed in the bridge member 530 thereby providing the surgeons with increased flexibility in positioning the spikes 540, however this is but one configuration and other configurations are envisioned.

In some examples, the geometry of the spikes that contacts the bone could be altered to be of a different geometry but achieve the same goal such as, for example, a hook, a slot, a pin, etc.

### Alignment Instrument

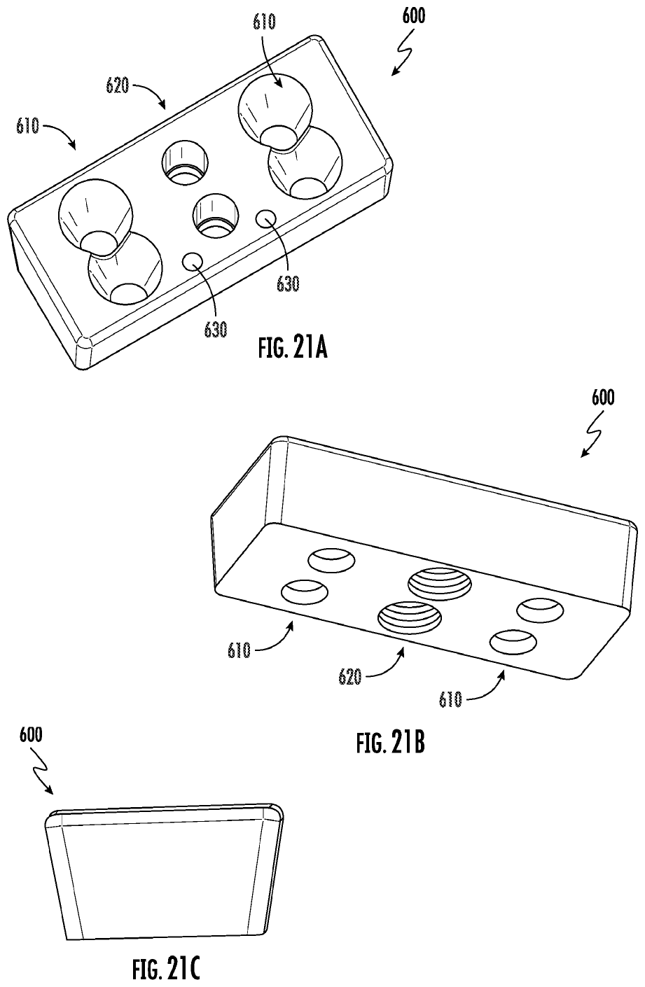

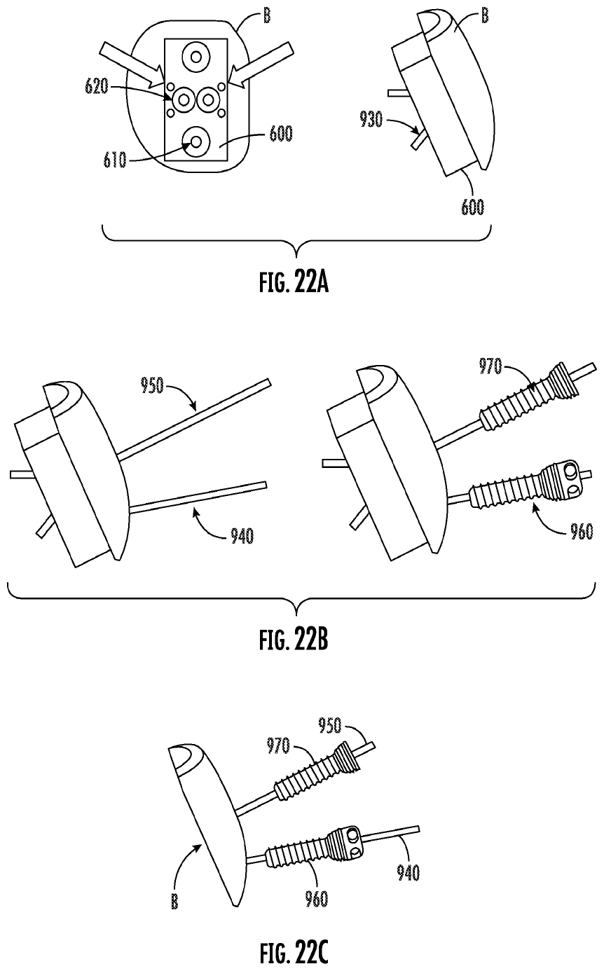

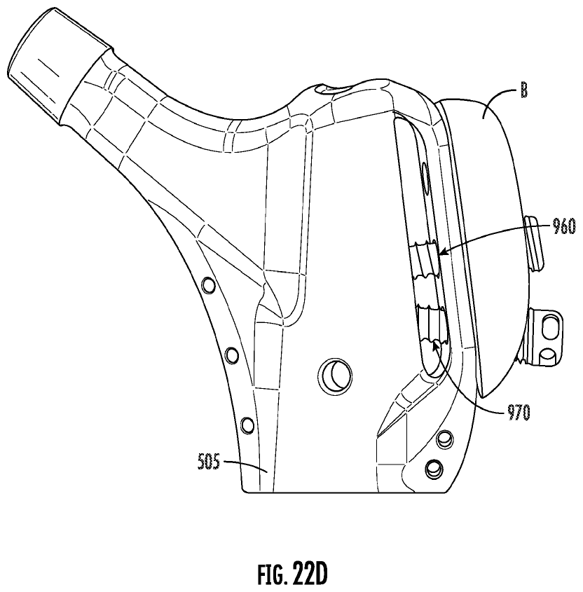

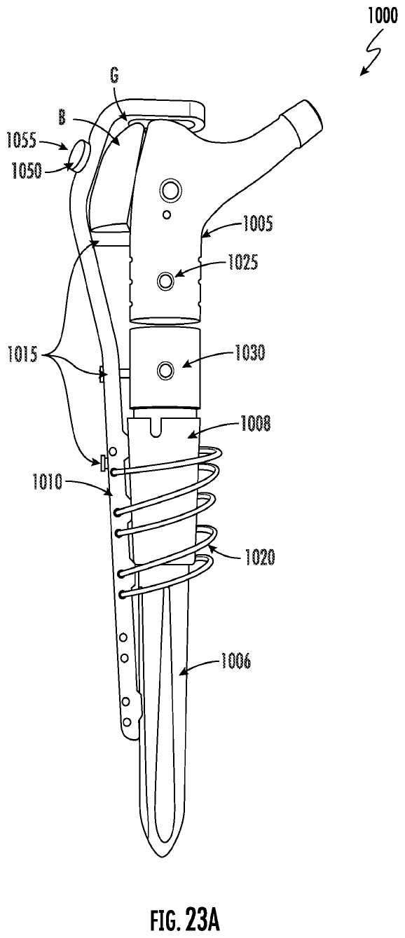

With reference to FIGS. 21A-21C and 22A-22D, in accordance with one or more features of the present disclosure that can be utilized in combination and/or separately from the other features disclosed herein, an alignment instrument 600 can be used to aid with alignment of the bone B as will be described in greater detail below. In use, the alignment instrument 600 can be temporarily coupled (e.g., pinned) to the bone B. Thereafter, the alignment instrument 600 can be used to predrill corresponding holes in the bone B to facilitate alignment with the holes formed in the bridge member 510 of the implant 505 (e.g., formation of the screw holes in the bone B).

In use, with the alignment instrument 600 positioned adjacent to the bone B, a drill having a minor diameter of a compatible fastener can be utilized. Thus arranged, access from the opposite side of the bone B can be provided to locate the holes in the bone B and pinning through to facilitate alignment of the bone B with the proximal femur replacement implant 505 (e.g., facilitates improved alignment of the proximal femur replacement implant 505 with the position of the anatomy).

In use, the alignment instrument 600 provides a solution for alignment by creating a structure in the anatomy (e.g., bone). In use, the alignment instrument 600 operates to match the inner diameter of the fastener and aligning a pin through the resected bone such that a fastener can be placed from the opposite side and the threads affix the fastener to the bone. This fastener can then be used as a stand-alone fixation mechanism, or affixed to a bone plate, or affixed with a cable saddle screw to provide a platform for cabling.

That is, as previously mentioned, the bridge member 510 of the proximal femur replacement implant 505 can include locking screw openings 522 and variable angled openings 524 to receive one or more fasteners. In use, the alignment instrument 600 includes openings 610 that correspond with the openings formed in the proximal femur replacement implant 505. In particular, in some examples, as illustrated, the alignment instrument 600 includes openings 610 that may be configured as a cone to mimic the fifteen-degree angle provided by the variable angled screw openings 524 formed in the bridge member 510 of the implant 505. In use, the openings 610 project the alignment to the resected bone. In use, this can be located using pins and either leaving the pins in to be located with cannulated screws from the opposite side of the resected bone, or removing the pins to allow for a non-cannulated screw from the opposite end of the resected bone.

In addition, the alignment instrument 600 may also include one or more holes 620 such as, for example, a pair of holes, centrally located, without an angle to locate the fasteners from the opposite end of the resected bone using the same means. In addition, the alignment instrument 600 may also include one or more holes 630 such as, for example, a pair of holes, at an angle to facilitate inserting one or more pins, guidewires, ligaments, etc. to temporary secure the alignment instrument 600 to the bone B to facilitate drilling of the screw openings.

Moreover, in use, the alignment instrument 600 could also be used by leaving the pins inside the resected bone, using a cannulated screw from the opposite side, and attaching to the proximal femur in that way.

In alternate examples, different variation of screws or pins could be used, the hole pattern could be varied, the number of connecting holes could vary, and it could be affixed to the resected bone in different ways.

With reference to FIGS. 22A-22D, an exemplary method of use will now be described. As illustrated, in use, the alignment instrument 600 can be positioned adjacent to the bone B. The various holes 610, 620, 630 in the alignment instrument 600 can be used to mimic the corresponding hole pattern and surface geometry of the implants and trials. For example, conical holes 610 may be provided to allow for a guide wire to be placed through the alignment instrument 600 at any angle up to fifteen-degrees in 360 degrees of rotation. In addition, one or more holes 620 (e.g., straight pin holes) may be provided in the alignment instrument 600 to allow for a guide wire to be pinned perpendicular to the implant/trial surface. In use, the alignment instrument 600 can be coupled to the bone using speed pins 930 inserted through holes 630.

That is, with the alignment instrument 600 positioned against the bone B, pins 930 can be inserted through holes 630 formed in the alignment instrument 600 to pin the bone B to the instrument 600. Once pinned in the desired location, guide wires 940, 950 may be inserted through holes 610 formed in the alignment instrument 600 and through the bone (e.g., through the preserved bone fragment B of the greater trochanter). Next, cannulated screws 960, 970 may be aligned using the guide wires 940, 950 from the opposite side of the instrument 600 and drilled through the bone fragment either at an angle 960 or perpendicular 970 relative to the implant and/or trial mating surface. Subsequently, the alignment instrument 600 may be removed from the preserved bone B.

Finally, intra-Operatively, the bone fragment B may then be secured to the bridge member 510 of the implant 505 with screws 960, 970 to secure the bone fragment B to the implant 505. These screws 960, 970 can be perpendicular 960 or at an angle 970 which fit into either the variable angled openings or locking screw openings formed in the bridge member 510 of the implant 505.

### Proximal Femur Replacement Implant Assembly

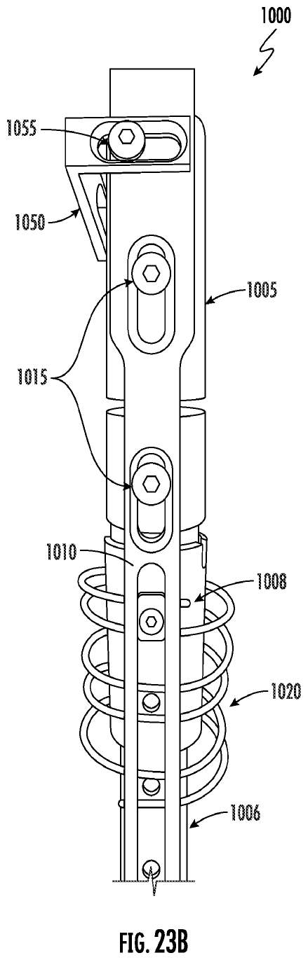

With reference to FIGS. 23A and 23B, an alternate example of a proximal femur replacement implant assembly 1000 in accordance with one or more features of the present disclosure is shown. The proximal femur replacement implant assembly 1000 is substantially similar to the proximal femur replacement implant assemblies previously described herein and may include one or more of the various features, elements, etc. as those previously described herein. As such, for purposes of brevity, some of the description of features is omitted herefrom.

As illustrated, the proximal femur replacement implant assembly 1000 includes a proximal femur replacement implant 1005 and a first or primary bone plate 1010 coupled thereto, although this is but one configuration. As illustrated, the proximal femur replacement implant 1005 may also include a stem portion 1006 arranged and configured to be received within an intramedullary canal of the patient's bone. In addition, the proximal femur replacement implant 1005 may include a sleeve 1008 such as, for example, a porous sleeve or augment, arranged and configured to be positioned over the stem 1006 for receipt within the patient's intramedullary canal. In some examples, the sleeve 1008 may include an opening 1030 such as, for example, a threaded hole, arranged and configured to receive a set screw or a locking screw for coupling to, for example, a secondary bone plate 1050, as will be described in greater detail below.

In the illustrated example, in use (e.g., when implanted), the first or primary bone plate 1010 is configured to wrap around the patient's greater trochanter B superiorly and laterally. In addition, the first or primary bone plate 1010 may be configured to be fixated, coupled, etc. to the proximal femur replacement implant 1005 superiorly and laterally.

In use, the first or primary bone plate 1010 can be coupled to the proximal femur replacement implant 1005 or to any one or more of the segment(s) joined to the proximal femur replacement implant 1005 by any suitable mechanism now known or hereafter developed. For example, as illustrated, the first or primary bone plate 1010 can be coupled to the proximal femur replacement implant 1005 or to any one or more of the segment(s) joined to the proximal femur replacement implant 1005 via one or more screws 1015. In addition, and/or alternatively, the first or primary bone plate 1010 may be configured to receive one or more cables 1020 for coupling the first or primary bone plate 1010 to the proximal femur replacement implant 1005 or to the implant system (e.g., cabling can be configured to pass through the first or primary bone plate 1010 and wraps around the proximal femur replacement implant 1005).

In some examples, the proximal femur replacement implant 1005 may include one or more through holes 1025. In use, the through holes 1025 formed in the proximal femur replacement implant 1005 may receive one or more cables, sutures, etc. to enable cabling or suturing through the proximal femur replacement implant 1005. In addition, and/or alternatively, the through holes 1025 may be utilized to couple to the secondary bone plate 1050.

As shown in the illustrated exemplary example, the proximal femur replacement implant assembly 1000 may also include a secondary bone plate 1050. In use, the secondary bone plate 1050 is arranged and configured to couple to the first or primary bone plate 1010 to wrap around the anterior side of the proximal femur replacement implant 1005 to form a “cage” around the patient's preserved greater trochanter bone B. As illustrated, the secondary bone plate 1050 may include a radiused surface thereon to enable variability of placement. In addition, and/or alternatively, as illustrated, in some embodiments, the secondary bone plate 1050 may include an elongated slot 1060 such as, for example, a radiused elongated slot 1060, to enable, allow, etc. angular variability of the secondary bone plate 1050 relative to the proximal femur replacement implant 1005.

The secondary bone plate 1050 may be coupled to the first or primary bone plate 1010 and/or the proximal femur replacement implant 1005 by any suitable coupling mechanism now known or hereafter developed including, for example, one or more fixation screws 1055.

Thus arranged, in use, the first or primary bone plate 1010 extends laterally and superiorly from the proximal femur replacement implant 1005. As such, the first or primary bone plate 1010 and the proximal femur replacement implant 1005 define a space or a gap G. In various examples, the surfaces of the proximal femur replacement implant 1005 including, without limitation, the outer surface thereof, and the surfaces of the first or primary bone plate 1010 may include one or more porous coatings. In use, by providing a plurality of porous coatings, the proximal femur replacement implant assembly 1000 (e.g., the proximal femur replacement implant 1005, the first or primary bone plate 1010, etc.) is configured to promote bone ingrowth from either or both sides of the proximal femur replacement implant assembly 1000. Thus arranged, in use, the gap G is arranged and configured to receive a portion of the bone B with the bone B in contact with the porous coatings of the proximal femur replacement implant 1005 and the first or primary bone plate 1010. In addition, in some examples, the secondary bone plate 1050 may be positioned anteriorly and may be coupled to the primary bone plate 1010 and/or the proximal femur replacement implant 1005 to form a cage about the bone B. Surfaces of the secondary bone plate 1050 may also include a porous coating to promote bone ingrowth.

In use, the geometry of the proximal femur replacement implant assembly 1000 provides surgeons with increased flexibility to facilitate coupling of the bone B such as, for example, a patient's greater trochanter to the proximal femur replacement implant assembly 1000. In use, the surgeon can elect to couple one or more bone plates to the proximal femur replacement implant 1005 with, for example, the bone B positioned between the proximal femur replacement implant 1005, the first or primary bone plate 1010, and the secondary bone plate 1050. Thus arranged, the bone B such as, for example, the patient's greater trochanter, can be reattached to the proximal femur replacement implant 1005 with sufficient constraint to allow for porous ingrowth and overall system longevity. In use, the proximal femur replacement implant assembly 1000 is configured to limit anterior/posterior and/or medial/lateral translation in a manner that retains the proximal/distal and medial/lateral constraints, while also minimizing micromotion to better accommodate porous ingrowth between the bone B and the replacement implant.

In conclusion, in accordance with various features disclosed herein, in use, when in the process of reattaching the greater trochanter to the proximal femur replacement implant assembly in surgery, anterior/posterior and medial/lateral translation can be minimized to retain the proximal/distal and medial/lateral constraints, while also minimizing micromotion to better accommodate porous ingrowth between the preserved bone and surgical implants. In use, this attachment mechanism allows a variety of uses including screw holes, variable angle screw holes, slots, etc. to allow for a synthetic ligament if no bone is able to be preserved. As such, the greater trochanter can be reattached to the proximal femur replacement implant assembly with sufficient constraint to allow for porous ingrowth and overall system longevity.

The various components including the proximal femur replacement implants, the primary bone plate (e.g., hook plate), the secondary bone plate, the customizable bone plates, etc. may be manufactured from any suitable biocompatible material now known or hereafter developed used to manufacture orthopedic implants. For example, the components may be made from cobalt chromium, stainless steel, titanium, oxidized zirconium, or other metal alloys. Alternatively, the components may be manufactured from standard polyethylene, cross-linked polyethylene, ultra-high molecular weight plastic, other plastics, ceramics, or a composite material.

In various examples, the various components may include a porous coating applied thereto for additional boney in-growth and/or fixation. In some examples, the porous coating may be printed, sprayed, or sintered onto the bone facing surfaces of the implants. For example, the porous coating may be formed by plasma spraying titanium, cobalt chrome, zirconium, oxidized zirconium, or stainless steel over the bone facing surfaces of the implants. Alternatively, the porous coating could be a hydroxyapatite or other similar coating known in the art to enhance bony ingrowth. In some examples, the entire implant could be coated. Alternatively, only sections of the implant may be coated. In addition, and/or alternatively, the implants may include one or more roughened surfaces to facilitate enhanced bony ingrowth. For example, the implants, or at least portions thereof, may include one or more macro-grooves.

The implants may be formed using any desired or appropriate methodologies or technologies now known or hereafter developed. For example, the implants may be manufactured using any now known or hereafter developed additive manufacturing technique. By way of some, non-limiting known techniques, the implants could be manufactured from selective laser sintering (SLS), direct metal laser sintering (DMLS), electron beam melting (EBM), selective laser melting (SLM), three-dimensional printing, or the like. For instance, in some examples, each of the implants may be formed as a monolithic or integral implant (including any porous or other in-growth promoting surfaces or materials). In some examples, portions of each implant may be formed and then additional in-growth materials, surfaces, and/or treatments could be added or applied to the implant. In other examples, an additive manufacturing technique such as, for example, electron beam melting methods or methods that use lasers to subtract or remove select portions of material from an initially solid material may be used. In other examples, portions or all of the implants can be formed using casting or other technologies or methods. In some examples, a non-porous implant may be formed using an additive manufacturing technique such as, for example, SLS technologies and subsequently that implants may be subjected to acid etching, grit blasting, plasma spraying (e.g., of titanium oxide or another metal to promote in-growth) or other treatments.

While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples, but that it has the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples, or configurations. Any example or feature of any section, portion, or any other component shown or particularly described in relation to various examples of similar sections, portions, or components herein may be interchangeably applied to any other similar example or feature shown or described herein. Additionally, components with the same name may be the same or different, and one of ordinary skill in the art would understand each component could be modified in a similar fashion or substituted to perform the same function.

Moreover, the following claims are hereby incorporated into this Detailed Description by this reference, with each claim standing on its own as a separate example of the present disclosure.

As used herein, an element or step recited in the singular and proceeded with the word “a” or “an” should be understood as not excluding plural elements or steps, unless such exclusion is explicitly recited. Furthermore, references to “one example” of the present disclosure are not intended to be interpreted as excluding the existence of additional examples that also incorporate the recited features.

The phrases “at least one”, “one or more”, and “and/or”, as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

1. A proximal femur replacement implant assembly arranged and configured to replace a head portion of a patient's proximal femur, the assembly comprising:
a proximal femur replacement implant arranged and configured to be coupled to, or inserted into an intramedullary canal of, a remaining portion of a patient's femur, the proximal femur replacement implant including a proximal end, a distal end, a first side surface, a second side surface, a medial side surface, a lateral side surface, and a bridge member spaced from the lateral side surface;
wherein the bridge member includes an implant facing surface, an outer surface opposite the implant facing surface, and a plurality of openings formed therein for receiving a plurality of fasteners.

2. The assembly of claim 1, wherein the plurality of openings include one or more locking screw openings and one or more variable angled openings.

3. The assembly of claim 1, wherein the bridge member is integrally and monolithically formed with the proximal femur replacement implant.

4. The assembly of claim 1, wherein the bridge member includes a porous surface formed on a portion thereof.

5. The assembly of claim 1, further comprising a bone plate and the plurality of fasteners for coupling the bone plate to the bridge member.

6. The assembly of claim 1, further comprising the plurality of fasteners, wherein at least one of the plurality of fasteners comprises a cable saddle screw including a threaded shaft and a head portion, the head portion including a plurality of transverse openings arranged and configured to enable a cable, a suture, or a ligament to pass therethrough.

7. The assembly of claim 6, wherein at least one of the plurality of openings formed in the bridge member is arranged and configured to engage the threaded shaft of the cable saddle screw.

8. The assembly of claim 1, wherein the bridge member includes one or more spikes or projections extending laterally therefrom to facilitate initial fixation.

9. The assembly of claim 1, wherein spacing the bridge member from the lateral side surface defines a gap between the lateral side surface of the proximal femur replacement implant and an inner surface of the bridge member, the gap arranged and configured to receive a portion of a bone therein.

10. A proximal femur replacement implant assembly arranged and configured to replace a head portion of a patient's proximal femur, the assembly comprising:
a proximal femur replacement implant arranged and configured to be coupled to, or inserted into an intramedullary canal of, a remaining portion of a patient's femur, the proximal femur replacement implant including a proximal end, a distal end, a first side surface, a second side surface, a medial side surface, and a lateral side surface;
a primary bone plate arranged and configured to be coupled to a patient's bone; and
a secondary bone plate arranged and configured to couple the primary bone plate to the proximal femur replacement implant.

11. The assembly of claim 10, wherein the proximal femur replacement implant includes one or more screw fixation holes formed in the first and second side surfaces of the proximal femur replacement implant, each of the one or more screw fixation holes arranged and configured to receive a fastener or screw for coupling the secondary bone plate to the first and second side surfaces of the proximal femur replacement implant.

12. The assembly of claim 11, wherein the first and second side surfaces of the proximal femur replacement implant each include a recessed surface, the one or more screw fixation holes being formed in the recessed surface, the recessed surfaces being arranged and configured to be recessed a sufficient distance to reduce the extent that the secondary bone plate extends from an outer surface of the proximal femur replacement implant.

13. The assembly of claim 11, wherein the secondary bone plate includes a first end, a second end, a central portion extending between the first and second ends, and a plurality of openings formed therein for receiving a fastener so that, in use:
the first end of the secondary bone plate is coupled to the first side surface of the proximal femur replacement implant via one or more fasteners passing through one of the openings formed in the first end of the secondary bone plate and into one of the screw fixation holes formed in the first side surface of the proximal femur replacement implant,
the central portion of the secondary bone plate extends across a surface of the primary bone plate; and
the second end of the secondary bone plate is coupled to the second side surface of the proximal femur replacement implant via one or more fasteners passing through one of the openings formed in the second end of the secondary bone plate and into one of the screw fixation holes formed in the second side surface of the proximal femur replacement implant.

14. The assembly of claim 10, wherein the primary bone plate includes a hook-shaped plate including one or more hooks formed on a proximal end thereof configured to engage a patient's proximal femur.

15. The assembly of claim 10, wherein the secondary bone plate is pressed-fitted to the primary bone plate.

16. A proximal femur replacement implant assembly arranged and configured to replace a head portion of a patient's proximal femur, the assembly comprising:
a proximal femur replacement implant arranged and configured to be coupled to, or inserted into an intramedullary canal of, a remaining portion of a patient's femur, the proximal femur replacement implant including a proximal end, a distal end, a first side surface, a second side surface, a medial side surface, and a lateral side surface; and
a customizable bone plate arranged and configured to be flexible, the bone plate arranged and configured to be coupled to a patient's bone and arranged and configured to couple to the proximal femur replacement implant.

17. The assembly of claim 16, wherein the bone plate includes a center hub and a plurality of arms extending from the center hub.

18. The assembly of claim 17, wherein the plurality of arms are flexible so that, in use, a surgeon can reposition each of the arms.

19. The assembly of claim 17, wherein the proximal femur replacement implant includes one or more screw fixation holes formed in the first and second side surfaces of the proximal femur replacement implant, each of the one or more screw fixation holes arranged and configured to receive a fastener or screw for coupling one of the plurality of arms to the first and second side surfaces of the proximal femur replacement implant.

20. (canceled)
